(19) United States
(12) Patent Application Publication
(10) Pub. No.: US 2023/0107927 A1
(43) Pub. Date: Apr. 6, 2023

(54) METHODS OF TREATING IATROGENIC AUTOIMMUNE COLITIS

(71) Applicant: First Wave Bio, Inc., Ann Arbor, MI (US)

(72) Inventor: Gary Glick, Ann Arbor, MI (US)

(21) Appl. No.: 17/802,663

(22) PCT Filed: Feb. 26, 2021
(86) PCT No.: PCT/US2021/019943

§ 371 Date: Aug. 26, 2022

Related U.S. Application Data
(60) Provisional application No. 62/983,049, filed on Feb. 28, 2020.

(51) Int. Cl. C07K 16/28; A61K 31/137; A61K 31/4245; A61K 45/06; A61P 1/00

(52) U.S. Cl. CPC C07K 16/2818; C07K 16/2827; A61K 31/137; A61K 31/4245; A61K 45/06; A61P 1/00; C07K 2317/76

(57) ABSTRACT

This disclosure features compounds and compositions for use in methods of treating iatrogenic autoimmune colitis in a subject in need thereof, e.g., iatrogenic autoimmune colitis induced by one or more chemotherapeutic agents (e.g., a chemotherapeutic immunomodulatory; e.g., an immune checkpoint inhibitor; e.g., an immune checkpoint inhibitor that targets CTLA-4). The methods Include administering to the subject one or more chemical entities including, but not limited to, sphingosine 1-phosphate (S1P) receptor modulators; Janus kinase (JAK) inhibitors; lanthionine synthetase C-like 2 (LANCL2) modulators; integrin modulators; and immunosuppressants (e.g., cyclosporine).

### CROSS REFERENCE TO RELATED APPLICATIONS

This application claims the benefit of U.S. Provisional Application No. 62/983,049, filed on Feb. 28, 2020, which is incorporated herein by reference in its entirety.

### TECHNICAL FIELD

This disclosure features compounds and compositions for use in methods of treating iatrogenic autoimmune colitis in a subject in need thereof, e.g., iatrogenic autoimmune colitis induced by one or more chemotherapeutic agents (e.g., a chemotherapeutic immunomodulator; e.g., an immune checkpoint inhibitor; e.g., an immune checkpoint inhibitor that targets CTLA-4). The methods include administering to the subject one or more chemical entities including, but not limited to, sphingosine 1-phosphate (S1P) receptor modulators; Janus kinase (JAK) inhibitors; lanthionine synthetase C-like 2 (LANCL2) modulators; integrin modulators; and immunosuppressants (e.g., cyclosporine).

### BACKGROUND

Ulcerative colitis (UC) and Crohn's disease (CD) are the predominant chronic, inflammatory bowel diseases (IBD) in humans. These disorders are autoimmune in nature and occur in the absence of infection. IBD affects up to 2,000,000 Americans (increasing ˜15% annually), and it is associated with unacceptably high rates of morbidity and mortality.

IBD occurs as the result of inappropriate immune responses in genetically susceptible individuals mediated by complex interactions between environmental stimuli, microbial factors, and the intestinal immune system. The hallmark of IBD is represented by excessive immune responses that mediate gastrointestinal tissue damage, either directly or through the release of soluble, pro-inflammatory mediators.

The use of checkpoint inhibitor therapy in chemotherapy can lead to immune-related adverse effects. Severe colitis has been reported in patients treated with cytotoxic T-lymphocyte-associated antigen-4 (CTLA-4) inhibitors, such as ipilimumab. In some cases, immunotherapy-induced colitis can become life-threatening and result in bowel perforation requiring surgical intervention. The foregoing is believed to be caused by ipilimumab's removal of immune regulation, specifically through the inactivation of cytotoxic T-lymphocyte-associated antigen 4 (CTLA-4). See, e.g., Delasos, et al., Case Reports in Oncological Medicine, Volume 2019, Article ID 9069354, 6 pages; https://doi.org/10.1155/2019/9069354.

WO 2017/040864 discloses, e.g., methods for treating iatrogenic autoimmune colitis, which include administering a mitochondrial uncoupling agent, e.g., niclosamide.

### SUMMARY

This disclosure features compounds and compositions for use in methods of treating iatrogenic autoimmune colitis in a subject in need thereof, e.g., iatrogenic autoimmune colitis induced by one or more chemotherapeutic agents (e.g., a chemotherapeutic immunomodulatory; e.g., an immune checkpoint inhibitor; e.g., an immune checkpoint inhibitor that targets CTLA-4). The methods include administering to the subject one or more chemical entities including, but not limited to, sphingosine 1-phosphate (S1P) receptor modulators; Janus kinase (JAK) inhibitors; lanthionine synthetase C-like 2 (LANCL2) modulators; integrin modulators; and immunosuppressants (e.g., cyclosporine).

In one aspect, disclosed herein is a method for treating iatrogenic autoimmune colitis in a subject in need thereof, the method comprising administering to the subject an effective amount of any one or more chemical entities described herein.

In some embodiments, the chemical entity or a pharmaceutically acceptable salt thereof is selected from the group consisting of:

sphingosine 1-phosphate receptor (S1P) modulators;

Janus kinase (JAK) inhibitors;

lanthionine synthetase C-like 2 (LANCL2) modulators;

integrin modulators;

immunosuppressant;

steroidal anti-inflammatory agents;

non-steroidal anti-inflammatory agents;

receptor-interacting protein kinase 1 (RIPK1) inhibitors;

receptor-interacting serine/threonine kinase 2 (RIPK2) inhibitors;

EP4 modulators;

toll-like receptor modulators;

phosphatidylcholine;

Smad7 modulators;

phosphodiesterase 4 (PDE4) modulators;

tumor progression locus 2 (TPL2) inhibitors;

tyrosine kinase 2 (TYK2) inhibitors;

TEC kinase inhibitors;

TIP60 inhibitors;

emoxypine and/or, a glutamate 2b (Glut2B or GluN2B) receptor antagonists;

pantetheinase inhibitors (VNN1, Vanin-1);

bile salt hydrolase (BSH) modulators;

aryl hydrocarbon receptor (AhR) agonists;

prolyl hydroxylases- (PHDs) inhibitor;

selective GABAA receptor agonist (δ-subunit containing GABAA receptors);

KEAP1-Nrf2 inhibitors;

human histamine 4 receptor (hH4R) inhibitors;

peptidylarginine deiminase inhibitors (PAD4);

tumor necrosis factor-α (TNF-α) inhibitors;

MEK/ERK inhibitors;

IRAK4 inhibitors;

PKC inhibitors;

CCR9 chemokine receptor inhibitors;

retinoid-related orphan receptor (ROR)γt inhibitors;

NF-kB activation inhibitors;

g protein-coupled bile acid receptor 1 (GPBAR1; TGR5) agonists;

TMEM16 (TMEM16A and/or TMEM16F) inhibitors other than niclosamide;

PPARγ modulators; and

5-aminolevulinic acid,

or a pharmaceutically acceptable salt thereof.

In some embodiments, the chemical entity or a pharmaceutically acceptable salt thereof is selected from the group consisting of:

sphingosine 1-phosphate receptor (S1P) modulators;

Janus kinase (JAK) inhibitors;

lanthionine synthetase C-like 2 (LANCL2) modulators;

integrin modulators;

immunosuppressant;

steroidal anti-inflammatory agents;

non-steroidal anti-inflammatory agents;

receptor-interacting protein kinase 1 (RIPK1) inhibitors;

EP4 modulators;

toll-like receptor modulators;

phosphatidylcholine;

Smad7 modulators;

phosphodiesterase 4 (PDE4) modulators;

tumor progression locus 2 (TPL2) inhibitors;

tyrosine kinase 2 (TYK2) inhibitors;

TEC kinase inhibitors; and

TIP60 inhibitors;

or a pharmaceutically acceptable salt thereof.

In some embodiments, the chemical entity or a pharmaceutically acceptable salt thereof is a sphingosine 1-phosphate receptor (S1P) modulator (e.g., agonist) or a pharmaceutically acceptable salt thereof. Non-limiting examples include: fingolimod; CS-0777; KKSM-07003 (KKSM-07005, KKSM-07016, SKY-59); AKP-11; CBP-307; BMS-986104; SYL-933 (SYL-933-P); cenerimod (e.g., ACT-334441); NIBR-785; BMS-520 (BMS-54) (Hou et al., Org. Process Res. Dev. 20(5): 989-995, 2016); GSK-2018682 (2018682, PPI-4621, PPI-4667, PPI-4667-P, PPI-4939, PPI-4955, or PPI-5955-P); GSK1842799 (PPI-4691); KRP-107; AMG-247 (also called AMG-277, AMG-369, and PRX-13038); ponesimod (ACT-128800, Actelion-2, R-3477, RG-3477); YP-005; mocravimod dihydrochloride (also called KNF-299, KRP-203, KRP-203-P prodrug, and mocravimod); SAR-247799; SEW2871; KRP203; siponimod (BAF-312); ozanimod (RPC 1063); ceralifimod (ONO-4641); ASP4058; GSK2018682; PF-462991 (also called PF-04629991 and PF-991); LAS-189913; LC-510201; LC-51-SPA; LC-510201; A-971432; ABT-363; OBT-893 (SH-BC-893); RP-1859 (RP-1865); ASP4085; BMS-986166; VPC-01091; CP-1050; amitriptyline; LX-2932; LX-2931 (LX-3305); KDS-1059; KSI-6666; ozanimod metabolite (e.g., RP-101074, RP-101442, RP-101988, RPC-101075, and RPC-1063); TASP-0251078 (TASP-0277308); TY-52156; amiselimod (e.g., MT-1303); NOX-S91 (NOX-S92, NOX-S93); EXEL-4541 (XL-541); VPC23019; etrasimod (e.g., APD-334 or APD-334 L-Arginine); NIBR-0213; SPG-104; BML-258; PF-543; NV-06 (idronoxil, phenoxidiol); SKI-349; B-5354a; B-5354b; B-5354c; F-12509A; VPC-94075; SCL-5081308 (SRX-224014); ABC-294640 (ABC-294735, ABC-747080, SKI-I, SKI-II, SKI-V, Yeliva®, or opaganib); SLR080811; AB-22; ONO-1266; oxfenmino hydrochloric acid; and ABT-413, or a pharmaceutically acceptable salt thereof. For example, the chemical entity or a pharmaceutically acceptable salt thereof can be etrasimod. As another example, the chemical entity or a pharmaceutically acceptable salt thereof can be ozanimod.

In some embodiments, the chemical entity or a pharmaceutically acceptable salt thereof is a Janus kinase (JAK) inhibitor. Non-limiting examples include: abrocitinib, baricitinib, BMS-986165, decemotinib (VX509); filgotinib; itacitinib; oclacitinib; peficitinib; PF-06651600; PF-06700841; R333 (R932333); R348 (R932348); ruxolitinib; solcitinib; TD-1473; TD-8236; TD-5202; TD-3504; tofacitinib (e.g., tofacitinib citrate); and upadacitinib. For example, the chemical entity or a pharmaceutically acceptable salt thereof can be selected from the group consisting of: TD-1473, tofacitinib, upadacitinib, filgotinib, PF-06651600, and PF-06700841. For example, the chemical entity or a pharmaceutically acceptable salt thereof can be TD-1473.

In some embodiments, the chemical entity or a pharmaceutically acceptable salt thereof is a lanthionine synthetase C-like 2 (LANCL2) modulator (e.g., agonist). Non-limiting examples include: BT-11, NX-13, and BT-13. For example, the chemical entity or a pharmaceutically acceptable salt thereof can be BT-11.

In some embodiments, the chemical entity or a pharmaceutically acceptable salt thereof is an integrin modulator (e.g., an α4 integrin modulator and/or an α4β7 integrin modulator). Non-limiting examples include: vedolizumab; natalizumab; etrolizumab; vatelizumab; PF-00547659; AJM-300; HCA2969 (carotegrast); firategrast; valategrast; R00270608; CDP-323; CT7758; GW-559090; ELND-004; PTG-100; and PN-10943. For example, the chemical entity or a pharmaceutically acceptable salt thereof can be vedolizumab.

In some embodiments, the chemical entity or a pharmaceutically acceptable salt thereof is an immunosuppressant. Non-limiting examples include: methotrexate, azathioprine (Imuran®), cyclosporine, tacrolimus, mycophenolate mofetil (Cellcept®), and cyclophosphamide (Cytoxan®), systemic or oral corticosteroids, rapamycin, FK-506, and interferon-gamma. For example, the chemical entity or a pharmaceutically acceptable salt thereof can be cyclosporine.

In some embodiments, the chemical entity or a pharmaceutically acceptable salt thereof can be selected from the group consisting of:
a steroidal anti-inflammatory agent (e.g, beclomethasone 17, budesonide prednisone, prednisolone, or beclometasone dipropionate;
a non-steroidal anti-inflammatory agent (e.g., 5-ASA);
a receptor-interacting protein kinase 1 (RIPK1) inhibitor (e.g., GSK2982772);
a receptor-interacting serine/threonine kinase 2 (RIPK2) inhibitors, e.g, ponatinib, sorafenib, regorafenib, gefitinib, erlotinib, GSK583 or GSK2983559 (e.g., see WO 2020139748); e.g., Compound 8, see, e.g., ACS Med. Chem. Lett. 2017, 8, 1048-1053;
an EP4 modulator (e.g., KAG-308);
a toll-like receptor (e.g., TLR4, TLR9) modulator (e.g., JKB-122, cobitolimod);
an Smad7 modulator (e.g., mongersen);
a phosphodiesterase 4 (PDE4) modulator (e.g., apremilast);
a tumor progression locus 2 (TPL2) inhibitor (e.g., GS-4875);
a tyrosine kinase 2 (TYK2) inhibitor (e.g., BMS-986165, PF-06700841, and PF-06826647);
a TEC kinase inhibitor (e.g., PF-06651600);
a TIP60 inhibitor (see, e.g., U.S. Patent Application Publication No. 2012/0202848)
emoxypine and/or glutamate 2b (Glut2B or GluN2B) receptor antagonists, e.g., ifenprodil, radiprodil, traxoprodil, rislenmdaz, eliprodil, Ro-25-6981, and BMT-108908, EVT-101, CP101-606, MK-0657, EVT-103, and AZD 6765 (Annual Reports in Medicinal Chemistry (2012) Volume 47: 94-103) (see, e.g., WO 2021003553);
pantetheinase inhibitors (VNN1, Vanin-1) (see, e.g., WO 2020102575, WO 2020114943, WO 2018228934, U.S. Pat. No. 10,364,255 B2);
bile salt hydrolase (BSH) modulators, e.g., Compound 7 (see, e.g., WO 2020231776);
aryl hydrocarbon receptor (AhR) agonists (e.g., indirubin, isatin (see, e.g., WO 20200227151), e.g., indole-3 carbinol, diindolylmethane, see, e.g., Int. J. Mol. Sci. 2018, 19, 385), (e.g., NPD-0414-2 or NPD-0414-24, see, e.g., Front. Pharmacol. 10:380. doi: 10.3389/fphar.2019.00380), e.g., PY109 or PY108, see, e.g., Sci. Adv. 6, eaay8230 (2020);
prolyl hydroxylases-(PHDs) inhibitors e.g., imidazolipyridinone compound, (see, e.g. WO 2020054788), (e.g., hypoxanthine compound, AKB-4924, (see, e.g., WO 2020054825);
selective GABAA receptor agonist (δ-subunit containing GABAA receptors), (e.g., gaboxadol, see, e.g., WO 2020041574);
KEAP1-Nrf2 inhibitors, e.g., sultam compounds (see, e.g., WO 2020041169 A2), e.g., small molecules (see, e.g., WO 2020150446);
human histamine 4 receptor (hH4R) inhibitors, e.g., heterocyclic compounds, (see, e.g., (WO 2019231270);
peptidylarginine deiminase inhibitors (PAD4), e.g., GSK199, and heterocyclic analogs (see, e.g., WO 2019161803, WO 2019077631, WO 2019058393, WO 2016185279);
tumor necrosis factor-α (TNF-α) inhibitors (see, e.g., WO 2019146773, WO 2015011331);
MEK/ERK inhibitors e.g., mebendazole (see, e.g., WO 2019121996)
IRAK4 inhibitors, e.g. 2-substituted indazole compounds (see, e.g., WO 2017207386, WO 2015193846); see, bicyclic heterocyclic compound (see, e.g., WO 2013042137), (see, e.g., WO2014058685); e.g., Cell Signal. 2015; 27(6):1039-1055. doi:10.1016/j.cellsig.2015.02.025); (see, e.g., Curr Top Med Chem, 2009, 9(8), 724-737. https://doi.org/10.2174/156802609789044407;
PKC inhibitors (see, e.g., WO 2017087318);
CCR9 chemokine receptor inhibitors e.g., CCX507, (see, e.g., WO 2016057424, also see, Immunology Letters, 151, (2013), 44-47; e.g., vercirnon (also known as Traficet-EN or CCX282), (see, e.g., Curr Gastroenterol Rep (2015) 17: 21); e.g., biarylsulfonamide compound (see, e.g., Bioorg. Med. Chem. Lett. 25 (2015) 3661-3664);
retinoid-related orphan receptor (ROR)γt inhibitors e.g., BI119, (see, e.g., Frontiers Immunol., 9, 2307, (2018), doi:10.3389/fimmu.2018.02307); (see, e.g., WO 2016039408, WO 2015090507, U.S. Pat. No. 8,389,739 B1);
NF-kB activation inhibitors (see, e.g., WO2015028976); e.g., 3-[(dodecylthiocarbonyl)-methyl]-glutarimide (DTCM-G) or dehydroxymethylepoxyquinomicin (DHMEQ), (see, e.g., International Journal of Molecular Medicine 37 (6), (2016): 1457-1464. https://doi.org/10.3892/ijmm.2016.2560); (see, e.g., Front. Pediatr. 6:317. doi: 10.3389/fped.2018.00317); e.g., fumarate compounds, (see, e.g., WO 2016127186, WO 2016133832);
g protein-coupled bile acid receptor 1 (GPBAR1; TGR5) agonists (e.g., pyridine compounds (see, e.g., WO 2017106818);
TMEM16 (TMEM16A and/or TMEM16F) inhibitors other than niclosamide;
PPARγ modulators, e.g., 3-(4-aminophenyl)-2-methoxypropionic acid (see, e.g., WO2017093444), see also, Front. Pharmacol. 11:730. doi: 10.3389/fphar.2020.00730;
5-aminolevulinic acid (see, e.g., WO2020221827).

In some embodiments, the chemical entity or a pharmaceutically acceptable salt thereof can be selected from the group consisting of:
a steroidal anti-inflammatory agent (e.g, beclomethasone 17, budesonide prednisone, prednisolone, or beclometasone dipropionate;
a non-steroidal anti-inflammatory agent (e.g., 5-ASA);
a receptor-interacting protein kinase 1 (RIPK1) inhibitor (e.g., GSK2982772);
an EP4 modulator (e.g., KAG-308);
a toll-like receptor (e.g., TLR4, TLR9) modulator (e.g., JKB-122, cobitolimod);
an Smad7 modulator (e.g., mongersen);
a phosphodiesterase 4 (PDE4) modulator (e.g., apremilast);
a tumor progression locus 2 (TPL2) inhibitor (e.g., GS-4875);
a tyrosine kinase 2 (TYK2) inhibitor (e.g., BMS-986165, PF-06700841, and PF-06826647);
a TEC kinase inhibitor (e.g., PF-06651600); and
a TIP60 inhibitor (see, e.g., U.S. Patent Application Publication No. 2012/0202848).

In some embodiments, the chemical entity is selected from the group consisting of: diphenoxylate/atropine, loperamide, infliximab, 6-mercaptopurine, AbGn-168H, ABX464, ABT-494, adalimumab, AJM300, alicaforsen, AMG139, anrukinzumab, apremilast, ATR-107 (PF0530900), autologous CD34-selected peripheral blood stem cells transplant, azathioprine, bertilimumab, BI 655066, BMS-936557, certolizumab pegol (Cimzia®), cobitolimod, CP-690,550, CT-P13, DIMS0150, E6007, E6011, etrasimod, etrolizumab, fecal microbial transplantation, figlotinib, fingolimod, firategrast (SB-683699) (formerly T-0047), GED0301, GLPG0634, GLPG0974, guselkumab, golimumab, GSK1399686, HMPL-004 (Andrographis paniculata extract), IMU-838, Interleukin 2 (IL-2), laquinimod, masitinib (AB1010), matrix metalloproteinase 9 (MMP 9) inhibitors (e.g., GS-5745), MEDI2070, mesalamine, methotrexate, mirikizumab (LY3074828), natalizumab, NNC 0142-0000-0002, NNC0114-0006, ozanimod, peficitinib (JNJ-54781532), PF-00547659, PF-04236921, PF-06687234, QAX576, RHB-104, rifaximin, risankizumab, RPC1063, SB012, SHP647, sulfasalazine, TD-1473, thalidomide, tildrakizumab (MK 3222), TJ301, TNF-Kinoid®, tofacitinib, tralokinumab, TRK-170, upadacitinib, ustekinumab, UTTR1147A, V565, vatelizumab, VB-201, vedolizumab, and vidofludimus.

In some embodiments, the method comprises administering an effective amount of the chemical entity, or a pharmaceutically acceptable salt thereof, to the GI tract of the subject.

In some embodiments, the method comprises locally administering an effective amount of the chemical entity, or a pharmaceutically acceptable salt thereof to the GI tract of the subject.

In some embodiments, the method comprises topically administering an effective amount of the chemical entity, or a pharmaceutically acceptable salt thereof to the GI tract of the subject.

Embodiments can include one or more of the following features.

The chemical entity can be a gut-restricted (non-systemic) chemical entity. The chemical entity can be an intestinal-restricted chemical entity (e.g., colon-restricted chemical entity).

The chemical entity can be administered orally. The chemical entity can be administered by tablet or pill.

The chemical entity can be administered rectally (e.g., via enema, rectal gel, rectal foam, rectal aerosol, or suppository). For example, the chemical entity can be administered via enema.

The chemical entity can be administered locally or topically.

The subject can be a human.

The condition can be iatrogenic autoimmune colitis induced by one or more chemotherapeutic agents.

At least one of the one or more chemotherapeutic agents can be a chemotherapeutic immunomodulator such as an immune checkpoint inhibitor. The immune checkpoint inhibitor can be an inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155.

The immune checkpoint inhibitor can be selected from the group consisting of: Urelumab, PF-05082566, MEDI6469, TRX518, Varlilumab, CP-870893, Pembrolizumab (PD1), Nivolumab (PD1), Atezolizumab (formerly MPDL3280A) (PDL1), MEDI4736 (PD-L1), Cosibelimab (CK-301) (PD-L1), Envafolimab (KN035) (PD-L1), Avelumab (PD-L1), BMS-986189 (PD-L1), INCB86550 (PD-L1), AUNP12 (PD-L1), PDR001 (PD1), BMS-986016, MGA271, Lirilumab, IPH2201, Emactuzumab, INCB024360, Galunisertib, Ulocuplumab, BKT140, Bavituximab, CC-90002, Bevacizumab, MNRP1685A, BI 754091 (PD1), Sasanlizumab (PF-06801591) (PD1), MEDI0680 (PD1), INCMGA00012 (MGA012) (PD1), Spartalizumab (PDR001) (PD1), Dostarlimab (TSR-042), (PD1), Camrelizumab (PD1), Retifanlimab (PD1), Tislelizumab (BGB-A317) (PD1), Toripalimab (Tuoyi; JS001) (PD1), JTX-4014 (PD1), AMP-224 (PD1), Sintilimab, Zimberelimab (AB122, GLS-010) (PD1), Balstilimab (AGN2034) (PD1), and MGA271 (anti-B7-H3).

In some embodiments, the immune checkpoint inhibitor can be selected from the group consisting of: Urelumab, PF-05082566, MEDI6469, TRX518, Varlilumab, CP-870893, Pembrolizumab (PD1), Nivolumab (PD1), Atezolizumab (formerly MPDL3280A) (PDL1), MEDI4736 (PD-L1), Avelumab (PD-L1), PDR001 (PD1), BMS-986016, MGA271, Lirilumab, IPH2201, Emactuzumab, INCB024360, Galunisertib, Ulocuplumab, BKT140, Bavituximab, CC-90002, Bevacizumab, and MNRP1685A.

The immune checkpoint inhibitor can be an inhibitor that targets CTLA-4.

The immune checkpoint inhibitor can be an antibody. The antibody can be ipilimumab, tremelimumab, BMS-986218, quavonlimab (MK-1308), Zalifrelimab (AGEN-1884), BMS-986249, BA-3071, or XTX101.

In some embodiments, the antibody is ipilimumab or tremelimumab.

The immune checkpoint inhibitor can be an inhibitor that targets PD1 or PD-L1. The immune checkpoint inhibitor can be selected from nivolumab, lambroizumab, and BMS-936559.

The methods can include administering more than one independently selected chemical entity (e.g., 2, 3, 4, 5, or 6 independently selected chemical entities).

The methods can further include administering one or more additional therapeutic agents.

In certain embodiments, the one or more therapeutic agents can be: budenoside; epidermal growth factor; sulfasalazine; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1 monoclonal antibodies; anti-IL-6 monoclonal antibodies (e.g., anti-IL-6 receptor antibodies and anti-IL-6 antibodies); growth factors; elastase inhibitors; pyridinyl-imidazole compounds; TNF antagonists as described herein; IL-4, IL-10, IL-13 and/or TGFbeta cytokines or agonists thereof (e.g., agonist antibodies); IL-11; glucuronide- or dextran-conjugated prodrugs of prednisolone, dexamethasone or budesonide; ICAM-1 anti sense phosphorothioate oligodeoxynucleotides (ISIS 2302; Isis Pharmaceuticals, Inc.); soluble complement receptor 1 (TP10; T Cell Sciences, Inc.); slow-release mesalazine; methotrexate; antagonists of platelet activating factor (PAF); ciprofloxacin; and/or lignocaine.

In some embodiments, the chemical entities described herein can provide targeted delivery of the chemical entities to certain regions of the GI tract (e.g., the colon, e.g., the ascending colon and/or the transverse colon and/or the distal colon (e.g., for treatment of an inflammatory bowel disease such as ulcerative colitis); e.g., the small bowel, e.g., the ileum (e.g., for treatment of an inflammatory bowel disease such as Crohn's disease)).

In some embodiments, administration (e.g., oral administration; e.g., rectal administration) of the chemical entities described herein to a subject produces a local concentration of the chemical entities in the GI tract (e.g., colon, e.g., supra; e.g., the small bowel, e.g., the ileum) of the subject that is higher than the concentration of the chemical entities in the plasma compartment of the subject, thereby, e.g., more efficiently providing the chemical entities to diseased tissue in the GI tract (e.g., supra) and reducing risks associated with high systemic exposure (e.g., toxicity) to the chemical entities. Moreover, the foregoing can potentially be achieved using a lower dosage of the chemical entities described herein.

In some embodiments, the methods and compositions described herein are suitable for use in combination therapy with various other therapeutic regimens (e.g., chemotherapy and/or radiation). In certain embodiments, the chemical entities and methods described herein can be used to treat side effects produced by such therapeutic regimens, e.g., inflammatory bowel diseases induced by chemotherapeutic immunomodulators, e.g., checkpoint inhibitors, which in some cases can be prohibitively severe.

### Definitions

To facilitate understanding of the disclosure set forth herein, a number of terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

The singular forms “a,” “an,” and “the” may refer to plural articles unless specifically stated otherwise.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “digestive tract” is understood to include the mouth, pharynx, esophagus, stomach, small intestine or small bowel (duodenum, jejunum, ileum), large intestine (colon (cecum, ascending colon, transverse colon, descending colon, sigmoid colon), rectum, and anus.

The term “oral cavity” is understood to include the mouth, the pharynx and the esophagus.

The term “gastrointestinal tract”, or “GI tract”, or gut is understood to include the stomach, small intestine or small bowel (duodenum, jejunum, ileum) and large intestine or colon (cecum, ascending colon, transverse colon, descending colon, sigmoid colon),

The term “acceptable” with respect to a formulation, composition or ingredient, as used herein, means having no persistent detrimental effect on the general health of the subject being treated.

“API” refers to an active pharmaceutical ingredient.

The terms “effective amount” or “therapeutically effective amount,” as used herein, refer to a sufficient amount of a chemical entity which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a chemical entity as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “pharmaceutically acceptable salt” refers to a formulation of a compound (e.g., one or more chemical entities described herein) that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glutamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a chemical entity described herein with other chemical components (referred to collectively herein as “excipients”), such as carriers, stabilizers, diluents, dispersing agents, suspending agents, and/or thickening agents. The pharmaceutical composition facilitates administration of the chemical entity to an organism. Multiple techniques of administering a chemical entity exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. Often, the beneficial effects that a subject derives from a therapeutic agent do not result in a complete cure of the disease, disorder or condition.

The details of one or more embodiments of the invention are set forth in the accompanying drawings and the description below. Other features, objects, and advantages of the invention will be apparent from the description and drawings, and from the claims.

### DETAILED DESCRIPTION

This disclosure features compounds and compositions for use in methods of treating iatrogenic autoimmune colitis in a subject in need thereof, e.g., iatrogenic autoimmune colitis induced by one or more chemotherapeutic agents (e.g., a chemotherapeutic immunomodulatory; e.g., an immune checkpoint inhibitor; e.g., an immune checkpoint inhibitor that targets CTLA-4). The methods include administering to the subject one or more chemical entities including, but not limited to, sphingosine 1-phosphate (S1P) receptor modulators; Janus kinase (JAK) inhibitors; lanthionine synthetase C-like 2 (LANCL2) modulators; integrin modulators; and immunosuppressants (e.g., Cyclosporine).

In one aspect, provided herein is a method for treating iatrogenic autoimmune colitis in a subject in need thereof, the method comprising administering to the subject an effective amount of one or more chemical entities described herein.

In some embodiments, the chemical entity or a pharmaceutically acceptable salt thereof is selected from the group consisting of:
sphingosine 1-phosphate receptor (S1P) modulators;
Janus kinase (JAK) inhibitors;
lanthionine synthetase C-like 2 (LANCL2) modulators;
integrin modulators;
immunosuppressant;
steroidal anti-inflammatory agents;
non-steroidal anti-inflammatory agents;
receptor-interacting protein kinase 1 (RIPK1) inhibitors;
receptor-interacting serine/threonine kinase 2 (RIPK2) inhibitors;
EP4 modulators;
toll-like receptor modulators;
phosphatidylcholine;
Smad7 modulators;
phosphodiesterase 4 (PDE4) modulators;
tumor progression locus 2 (TPL2) inhibitors;
tyrosine kinase 2 (TYK2) inhibitors;
TEC kinase inhibitors;
TIP60 inhibitors;
emoxypine and/or, a glutamate 2b (Glut2B or GluN2B) receptor antagonists
pantetheinase inhibitors (VNN1, Vanin-1);
bile salt hydrolase (BSH) modulators);
aryl hydrocarbon receptor (AhR);
prolyl hydroxylases-(PHDs) inhibitors;
selective GABAA receptor agonist (δ-subunit containing GABAA receptors);
KEAP1-Nrf2 inhibitors;
human histamine 4 receptor (hH4R) inhibitors;
peptidylarginine deiminase inhibitors (PAD4);
tumor necrosis factor-α (TNF-α) inhibitors;
MEK/ERK inhibitors;
IRAK4 inhibitors;
PKC inhibitors;
CCR9 chemokine receptor inhibitors;
retinoid-related orphan receptor (ROR)γt inhibitors;
NF-kB activation inhibitors; inhibitors of NF-kB activation, such as fumarate compounds;
g protein-coupled bile acid receptor 1 (GPBAR1; TGR5) agonists;
PPARγ modulators;
5-aminolevulinic acid,

or a pharmaceutically acceptable salt thereof.

### Chemical Entities

In some embodiments, it is advantageous when the resultant systemic bioavailability of the administered chemical entity is relatively low, and the resultant local bioavailability of the administered chemical entity is relatively high. The foregoing can be achieved, for example, by selecting chemical entities having a relatively low oral bioavailability (F), wherein:

F=Fa×Fg×Fh

in which Fa=fraction absorbed; Fg=fraction escaping gut metabolism; and Fh=fraction escaping hepatic metabolism (see Filipski, K. J., et al., Current Topics in Medicinal Chemistry, 2013, 13, 776-802). As the skilled artisan will appreciate, the degree of oral bioavailability can be influenced by various physicochemical attributes, such as molecular weight (“MW”), log P, number of hydrogen bond donors (“HBD”), number of hydrogen bond acceptors (“HBA”), number of rotatable bonds (“RB”), and polar surface area (“PSA”). It has been recognized that good oral bioavailability is typically observed in compounds having the following attributes: MW≤500, Log P≤5, HBD≤5, HBA≤10, rotatable bonds (RB)≤10, PSA≤140 (Id.). Accordingly, a non-limiting strategy for designing and selecting chemical entities having a relatively low oral bioavailability (F) can include selecting physicochemical attributes that confer properties outside of the preferred oral drug space (Id.).

In some embodiments, the chemical entity, or a pharmaceutically acceptable salt thereof, is a gut-restricted (non-systemic) chemical entity. As used herein, such chemical entities refer to those that selectively target one or more organs in the gut or GI tract and provide a relatively low exposure in systemic compartments (e.g., <50 ng/mL, <40 ng/mL, <30 ng/mL<20 ng/mL, <10 ng/mL<5 ng/mL). In certain embodiments, the gut-restricted (non-systemic) chemical entity is impermeable to the gut epithelia (e.g., less than 50%, less than 45%, less than 40%, less than 35%, less than 30%, less than 25%, less than 20%, less than 15%, less than 10%, less than 9%, less than 8%, less than 7%, less than 6%, less than 5%, less than 4%, less than 3%, less than 2%, less than 1%, less than 0.5%, less than 0.1% of the chemical entity crosses the intestinal lining).

In certain embodiments, the chemical entity, or a pharmaceutically acceptable salt thereof, is an intestinal-restricted chemical entity.

In certain embodiments, the chemical entity is a colon-restricted chemical entity.

Sphingosine 1-Phosphate Receptor (S1P) Modulators

In some embodiments, the chemical entity is a sphingosine 1-phosphate receptor (S1P) modulator.

In some embodiments, a S1P modulator is a S1P agonist. For example, a S1P agonist can be a small molecule (e.g., less than 900 daltons), a peptide, or a fusion protein. In some embodiments, the S1P agonist is a non-selective S1P1 agonist (e.g., fingolimod).

In some embodiments, a S1P1 modulator is a S1P antagonist. For example, a S1P antagonist can be an inhibitory nucleic acid, an antibody or fragment thereof, a fusion protein, or a small molecule (e.g., less than 900 daltons). In some embodiments, the inhibitory nucleic acid is a small interfering RNA or an antisense molecule.

Non-limiting examples of S1P modulators can include: fingolimod; CS-0777; KKSM-07003 (KKSM-07005, KKSM-07016, SKY-59); AKP-11; CBP-307; BMS-986104; SYL-933 (SYL-933-P); cenerimod (e.g., ACT-334441); NIBR-785; BMS-520 (BMS-54) (Hou et al., Org. Process Res. Dev. 20(5): 989-995, 2016); GSK-2018682 (2018682, PPI-4621, PPI-4667, PPI-4667-P, PPI-4939, PPI-4955, or PPI-5955-P); GSK1842799 (PPI-4691); KRP-107; AMG-247 (also called AMG-277, AMG-369, and PRX-13038); ponesimod (ACT-128800, Actelion-2, R-3477, RG-3477); YP-005; mocravimod dihydrochloride (also called KNF-299, KRP-203, KRP-203-P prodrug, and mocravimod); SAR-247799; SEW2871; KRP203; siponimod (BAF-312); ozanimod (RPC 1063); ceralifimod (ONO-4641); ASP4058; GSK2018682; PF-462991 (also called PF-04629991 and PF-991); LAS-189913; LC-510201; LC-51-SPA; LC-510201; A-971432; ABT-363; OBT-893 (SH-BC-893); RP-1859 (RP-1865); ASP4085; BMS-986166; VPC-01091; CP-1050; amitriptyline; LX-2932; LX-2931 (LX-3305); KDS-1059; KSI-6666; ozanimod metabolite (e.g., RP-101074, RP-101442, RP-101988, RPC-101075, and RPC-1063); TASP-0251078 (TASP-0277308); TY-52156; amiselimod (e.g., MT-1303); NOX-S91 (NOX-S92, NOX-S93); EXEL-4541 (XL-541); VPC23019; etrasimod (e.g., APD-334 or APD-334 L-Arginine); NIBR-0213; SPG-104; BML-258; PF-543; NV-06 (idronoxil, phenoxidiol); SKI-349; B-5354a; B-5354b; B-5354c; F-12509A; VPC-94075; SCL-5081308 (SRX-224014); ABC-294640 (ABC-294735, ABC-747080, SKI-I, SKI-II, SKI-V, Yeliva®, or opaganib); SLR080811; AB-22; ONO-1266; oxfenmino hydrochloric acid; ABT-413; and variants thereof. Further examples of S1P modulators include those described in WO 2019/118778, which is incorporated herein by reference in its entirety.

In some embodiments, the S1P modulator is etrasimod, ozanimod, or variants thereof. In certain embodiments, the S1P modulator is etrasimod. In certain embodiments, the S1P modulator is ozanimod. In certain embodiments, the S1P modulator is an ozanimod metabolite, such as RP-101074, RP-101442, RP-101988, RPC-101075, and RPC-1063.

In some embodiments, the S1P modulator is a peptide (e.g., R-002L103 (R-002L106)). In certain embodiments, the S1P modulator is a S1P1 agonist and a S1P3 agonist (e.g., R-002L103). In some embodiments, the S1P modulator is an ApoM-Fc engineered fusion protein (Swendeman et al, Sci. Signal 10(492): eaal2722, 2017). Non-limiting examples of peptides that modulate S1P include those described in WO 2019/118778, which is incorporated herein by reference in its entirety.

In some embodiments, the S1P modulator is an inhibitory nucleic acid (e.g., antisense nucleic acid). Non-limiting examples of nucleic acid that modulate S1P include those described in WO 2019/118778, which is incorporated herein by reference in its entirety.

In some embodiments, the S1P modulator is an antibody or an antigen-binding fragment/portion thereof (e.g., a Fab or a scFv). In some embodiments, the S1P modulator is a humanized antibody, a chimeric antibody, a multivalent antibody, or a fragment thereof. In some embodiments, the S1P modulator is a monoclonal antibody. In some embodiments, the S1P modulator is a humanized monoclonal antibody. In some embodiments, the S1P modulator is an antibody or an antigen-binding fragment/portion thereof (e.g., a Fab or a scFv) that is a S1P antagonist. Further non-limiting examples of antibodies and antigen-binding fragment/portion thereof that modulator S1P activities include those described in WO 2019/118778, which is incorporated herein by reference in its entirety.

In some embodiments, the S1P modulator is an ApoM-Fc engineered fusion protein (Swendeman et al, Sci. Signal 10(492): eaal2722, 2017).

In some embodiments, a S1P modulator selectively targets S1P1, S1P4 and/or S1P5. In certain embodiments, the S1P modulator is an S1P1 modulator. In some embodiments, the S1P1 modulator modulates the activity of sphingosine 1-phosphate phosphatase 1. In certain embodiments, the S1P modulator agonizes the activity of S1P1 (e.g., LAS-189913). In some embodiments, the S1P modulator is a sphingosine 1-phosphate phosphatase 2 inhibitor (Huang et al., FASEB J 30(8): 2945-2958, 2016). In some embodiments, the S1P modulator can modulate the activity and/or expression of S1P3 (e.g., a S1P1/S1P3 agonist). In some embodiments, the S1P1 modulator can modulate the activity and/or expression of S1P2 (e.g., a S1P1/S1P2 agonist). In some embodiments, the S1P modulator can modulate the activity and/or expression of S1P5 (e.g., a S1P5 agonist) (e.g., LC-51-SPA, LC-510201, A-971432, ABT-363, ozanimod (RPC-1063 or RPC1063 HCl), ceralifimod (ONO-4641), siponimod (BAF-312), OBT-893 (SH-BC-893), or RP-1859 (RP-1865)). In some embodiments, the S1P modulator is a S1P1 agonist and a S1P3 antagonist (e.g., VPC-01091 (Zhu et al., J. Med. Chem. 50: 6428-6435, 2007)).

In some embodiments, the S1P modulator is a partial agonist. In some embodiments, the S1P modulator is a sphingosine 1 phosphate lyase inhibitor. In some embodiments, the S1P modulator is a sphingosine kinase 1 inhibitor (e.g., SPG-104, BML-258, PF-543, NV-06 (idronoxil, phenoxidiol), or SKI-349). In some embodiments, the S1P modulator is a sphingosine kinase 2 inhibitor (e.g., SCL-5081308 (SRX-224014). In some embodiments, the S1P modulator is a sphingosine kinase ½ inhibitor. In some embodiments, the S1P modulator is a sphingosine-1-phosphate receptor 2 (S1P2) antagonist (e.g., AB-22, ONO-1266). In some embodiments, the SIP modulator targets S1P2 and is an EDG5 antagonist. In some embodiments, the S1P modulator is a sphingosine-1-phosphate receptor 3 (S1P3) antagonist. In some embodiments, a S1P modulator can also be a cannabinoid receptor antagonist (e.g., oxfenmino hydrochloric acid).

In some embodiments, the S1P modulator is phosphorylated in vivo (e.g., following administration to a subject), and thereafter, resembles naturally-occurring sphingosine-1-phosphate. In some embodiments, a S1P modulator reduces immune cell (e.g., T cells, macrophages, neutrophils, and/or B cells) migration and/or immune cell (e.g., T cell, macrophage, neutrophils, and/or B cells) differentiation and/or proliferation. In some embodiments, a S1P1 modulator decreases inflammation in a subject following administration. In some embodiments, a S1P modulator increases vasoconstriction, fibrosis, and cell proliferation in a subject following administration. In some embodiments, the S1P modulator binds to S1P1, is internalized and activates intracellular AKT and ERK signaling pathways. In some embodiments, the S1P modulator reduces intracellular calcium ion mobilization (e.g., cenerimod). In some embodiments, the S1P modulator reduces vascular permeability, reduces expression of one or more pro-inflammatory cytokines (e.g., one or more of IL-6, 11-17, IL-12/IL-23 p40, CCL2, and TNFa), and/or reduces expression of myeloperoxidase levels. In some embodiments, the S1P modulator reduces neutrophil infiltration. In some embodiments, a S1P modulator reduces migration of lymphocytes from lymph nodes. In some embodiments, a S1P modulator reduces the release of inflammatory cytokines, reduces organ and/or tissue damage, or maintains immune surveillance.

JAK Inhibitors

In some embodiments, the JAK inhibitor is a small molecule, an antibody, a peptide, a peptide fragment or a nucleic acid.

Non-limiting examples of JAK inhibitors include: abrocitinib, baricitinib, BMS-986165, decemotinib (VX509); filgotinib; itacitinib; oclacitinib; peficitinib; PF-06651600; PF-06700841; R333 (R932333); R348 (R932348); ruxolitinib; solcitinib; TD-1473; TD-3504; TD-8236; TD-5202; tofacitinib (e.g., tofacitinib citrate); upadacitinib.

Non-limiting examples of JAK inhibitors also include: R256, R333, INCB047986, INCB16562, NVP-BSK805, TD-8236, TD-5202, peficitinib (ASP015K, JNJ-54781532), tofacitinib (Xeljanz®, Jakvinus®, CP-690, 500), cucurbitacin I (JSI-124), CHZ868, or a variant thereof.

Further non-limiting examples of JAK inhibitors include those described in WO 2018/112245 and WO 2019/246273, each of which is incorporated herein by reference in its entirety.

In some embodiments, the JAK inhibitor is a pan-JAK inhibitor (e.g., 3 methylthespesilactam (Li et al., Biochem. Pharmacol. 86(10): 1411-8, 2013)). As used herein, the term “pan-JAK inhibitor” is a chemical entity that has an IC₅₀ of about 500 nM to 4 μM (e.g., about 500 nM to about 2 μM) for each of human JAK1, human JAK2, and human JAK3 isoforms, when the IC₅₀ is determined for each of wildtype human JAK1, wildtype human JAK2, and wildtype human JAK3 using similar assay conditions (e.g., the same assay conditions). In some embodiments, a pan-JAK inhibitor can be a chemical entity that has an IC₅₀ for wildtype human JAK1, wildtype human JAK2, and wildtype human JAK3 that are within ±10% of each other, when each of the IC₅₀ values is assays under similar assay conditions (e.g., the same assay, e.g., the human wildtype JAK1, wildtype human JAK2, and wildtype human JAK3 assay described in Kim et al., J. Med. Chem. 58(18):7596-5602, 2015). In some embodiments, the pan-JAK inhibitor is tofacitinib (Xeljanz®, Jakvinus®, tasocitinib, CP-690550; cerdulatinib (PRT2070); Pyridone 6; PF-06263276; JAK inhibitor 1 (CAS 457081-03-07; JAKi); baricitinib (Olumiant; LY3009104; INCB-28050); or variants thereof.

In some embodiments, the JAK inhibitor is a JAK1 inhibitor (e.g., GSK2586184, oclacitinib (PF03394197, Apoquel®), upadacitinib (ABT494), INCB039110, PF04965842, or SAR-20347). In certain embodiments, the JAK1 inhibitor is a selective JAK1 inhibitor. As used herein, the term “selective JAKI inhibitor” means a chemical entity that has an IC₅₀ for wildtype human JAK1 that is at least 10-fold (e.g., at least 20-fold) lower than each of the IC₅₀ for wildtype human JAK2 and the IC₅₀ for wildtype human JAK3 when measured using similar assay conditions (e.g., the same assay, e.g., the human wildtype JAK1, wildtype human JAK2, and wildtype human JAK3 assay described in Kim et al., J. Med. Chem. 58(18):7596-5602, 2015). In some embodiments, the JAKI inhibitor is (31S,4R)-3-ethyl-4-(3H-imidazo[1,2-a]pyrrolo[2,3-e]pyrazin-8-yl)-N-(2,2,2-trifluoroethyl)pyrrolidine-1-carboxamide as disclosed in international patent application PCT/US2014/062145, incorporated by reference herein in its entirety).

In some embodiments, the JAK inhibitor is a JAK2 inhibitor (e.g., CEP-33779, fedratinib (TG101348, SAR302503); lestaurtinib (CEP-701); AC-430; pacritinib (SB 1518); BMS-911543; XL019; INCB039110; Gandotinib® (LY-2784544); R723; Z3 (Sayyah et al., Mol. Cancer. Ther. 7(8):2308-2318, 2008); or a variant thereof). In certain embodiments, the JAK2 inhibitor is a selective JAK2 inhibitor. As used herein, the term “selective JAK2 inhibitor” means a chemical entity that has an IC₅₀ for wildtype human JAK2 that is at least 10-fold (e.g., at least 20-fold) lower than each of the IC₅₀ for wildtype human JAK1 and the IC₅₀ for wildtype human JAK3 when measured using similar assay conditions (e.g., the same assay, e.g., the human wildtype JAK1, wildtype human JAK2, and wildtype human JAK3 assay described in Kim et al., J. Med. Chem. 58(18):7596-5602, 2015).

In some embodiments, the JAK inhibitor is a JAK3 inhibitor (e.g., decernotinib (VX-509), R348 or a variant thereof). In certain embodiments, the JAK3 inhibitor is a selective JAK3 inhibitor. As used herein, the term “selective JAK3 inhibitor” means a chemical entity that has an IC₅₀ for wildtype human JAK3 that is at least 10-fold (e.g., at least 20-fold) lower than each of the IC₅₀ for wildtype human JAK2 and the IC₅₀ for wildtype human JAK1 when measured using similar assay conditions (e.g., the same assay, e.g., the human wildtype JAK1, wildtype human JAK2, and wildtype human JAK3 assay described in Kim et al., J. Med. Chem. 58(18):7596-5602, 2015).

In some embodiments, the JAK inhibitor is a JAK1 and JAK2 inhibitor (JAK1/JAK2 inhibitor). In certain embodiments, the JAK1 and JAK2 inhibitor is ruxolitinib (Jakafi®, Jakavi®, INCB018424), baricitinib (INCB028050, LY3009104), AZD1480, filgotinib (GLPG0634, G146034), momelotinib (GS-0387, CYT387), or a variant thereof.

In some embodiments, the JAK inhibitor is a JAK1 and JAK3 inhibitor. In certain embodiments, the JAK inhibitor is a selective JAK1/JAK3 inhibitor. As used herein, the term “selective JAK1/JAK3 inhibitor” means a chemical entity that has an IC₅₀ for wildtype human JAK1 and wildtype human JAK3, that are each at least 5-fold (e.g., at least 10-fold or at least 20-fold) lower than the IC₅₀ for wildtype human JAK2, when the IC₅₀ is determined for each of wildtype human JAK1, wildtype human JAK2, and wildtype human JAK3 using similar assay conditions (e.g., the same assay, e.g., the human wildtype JAK1, wildtype human JAK2, and wildtype human JAK3 assay described in Kim et al., J. Med. Chem. 58(18):7596-5602, 2015). In some embodiments, the selective JAK1/JAK3 inhibitor is ZM 39923; peficitinib (ASP015K; JNJ-54781532); or variants thereof.

In some embodiments, the JAK inhibitor is a TYK2 inhibitor. In some embodiments, the TYK2 inhibitor is Ndi-031301 (Akahane et al., Blood 128: 1596, 2016); BMS-986165; SAR-20347; tyrphostin A1; a triazolopyridine compound as disclosed in US 2013/0143915 (which is incorporated herein by reference in its entirety); or a variant thereof.

In some embodiments, the JAK inhibitor is a gut-selective JAK inhibitor, such as TD-1473.

Additional examples of JAK inhibitors that are small molecules are described in, e.g., Furomoto et al., BioDrugs 27(5):431-438, 2013; O' Shea et al., Ann. Rheum. Dis. 72(2):iil 11-ii-115, 2013; Sonbol et al., Ther. Adv. Hematol. 4(1): 15-35, 2013; Tanaka et al. (2015) J. Biochem. 158(3): 173-179, 2015; and Virtanen et al., BioDrugs 2019, 33, 15-32.

Lanthionine Synthetase C-Like Protein 2 (LANCL2) Modulators

In some embodiments, the chemical entity is a modulator of lanthionine synthetase C-like protein 2 (LANCL2). In some embodiments, the chemical entity activates (e.g., agonizes) LANCL2. Non-limiting examples of LANCL2 modulators include BT-11, NX-13 (see U.S. Pat. No. 10,487,057), ABA (see US 2019/0160100A1), and BT-13. Non-limiting examples of LANCL2 modulators also include BT-63, BT-110, and INT-10. Further non-limiting examples of LANCL2 modulators include those described in U.S. Pat. Nos. 9,839,635; 10,487,057; US 2019/0160100; and WO 2016/064445, each of which is incorporated herein by reference in its entirety.

Integrin Modulators

In some embodiments, the chemical entity is an integrin modulator. In certain embodiments, the chemical entity is an integrin inhibitor. Non-limiting examples of integrin inhibitors include: vedolizumab; natalizumab; etrolizumab; vatelizumab; PF-00547659; AJM-300; HCA2969 (carotegrast); firategrast; valategrast; R00270608; CDP-323; CT7758; GW-559090; ELND-004; PTG-100; PN-10943; and variants (e.g., prodrugs, biosimilars) thereof. Further non-limiting examples of integrin modulators include those described in WO 2019/245455, which is incorporated herein by reference in its entirety.

In some embodiments, the integrin modulator is an inhibitory nucleic acid. In some embodiments, the integrin modulator is a peptide (e.g., a cyclic peptide or acyclic peptide). In some embodiments, the integrin modulator is a peptidomimetic. In some embodiments, the integrin modulator is a disintegrin. In some embodiments, the integrin modulator is a small molecule. Non-limiting examples of integrin modulating inhibitory nucleic acids, peptides, peptidomimetics, disintegrins, and small molecules include those described in WO 2019/246455, which is incorporated herein by reference in its entirety.

In some embodiments, the integrin modulator is a RGD(ArgGly Asp)-mimetic antagonist such as tirofiban. In some embodiments, the integrin modulator is an α4 antagonist such as frategrast or AJM-300. In some embodiments, the integrin modulator is α4β1 antagonist (e.g., IVL745, BIO-1211, HMR 1031, valategrast (R411), GW559090X, TR14035). In some embodiments, the integrin modulator is an α4β7 antagonist (e.g., AJM300 or carotegrast). In some embodiments, the integrin modulator is a dual α4β1/α4β7 antagonist (e.g., AJM300 or carotegrast). In some embodiments, the integrin modulator is αvβ3 antagonist (e.g., L0000845704, SB273005). In some embodiments, the integrin modulator is α5β1 antagonist (e.g., JSM6427). In some embodiments, the integrin modulator is GLPG0974, MK-0429, JSM-6427, or a variant thereof. In some embodiments, the integrin modulator targets a β2 integrin, such as SAR-118 (SAR1118) or BMS-587101.

In some embodiments, the integrin modulator is selective for inhibition of integrin α4β7 interaction with MAdCAM-1 relative to inhibition of α4β1 with VCAM-1. For example, in some embodiments, the integrin modulator is one of the integrin inhibitors disclosed in US 2005/0209232; WO 2005/077914; WO 2005/077915; WO 2017/135471; WO 2017/135472; Gong et al, J. Med. Chem., 49:3402-3411 (2006); Gong et al., Bioorg. Med. Chem. Lett., 18: 1331-1335 (2008); Sidduri et al, Bioorg. Med. Chem. Lett., 23: 1026-1031 (2013); or Xu et al, Bioorg. Med. Chem. Lett., 23:4370-4373 (2013), each of which are incorporated by reference in their entireties herein.

In some embodiments, the integrin modulator is a (37 inhibitor. For example, in some embodiments, the integrin modulator is one of the integrin inhibitors disclosed in WO 2000/30681 or Callier Dublanchet et al., 220^{th} ACS Meeting (2000) Washington D.C., MEDI 142.

In some embodiments, the integrin modulator is an α4 inhibitor. Non-limiting examples include ELND-004 (Elan Corp. plc), ELND-002 (Elan Corp. plc), ET-3764 (Encycle Therapeutics Inc.), E-6007 (Eisai Co Ltd), ER-464195-01 (EA Pharma Co. Ltd.), HCA-3551 (EA Pharma Co. Ltd.), DW-908e (PS-181895; PS-460644; PS-489655; PS-969819; Pharmacopeia Inc.), AVA-4746 (TBC-3342; TBC-4746; Encysive/Schering-Plough), GW-559090 (GlaxoSmithKline plc), TRK-170 (Toray Industries Inc.), TRK-720 (Toray Industries Inc.), MK-0617 (Merck), MK-0668 (Merck), CT-737 (Elan/Wyeth), CT-747 (Elan/Wyeth), CT-757 (Elan/Wyeth), CT-767 (Elan/Wyeth), SB-683698 (TR-14035; TR-9109; Tanabe Seiyaku Co Ltd), R-1541 (Roche Holding Co.), and CP-664511 (Pfizer); and pharmaceutically acceptable salts thereof.

In some embodiments, the integrin modulator is an integrin alpha-L (ITGAL) inhibitor, such as an integrin inhibitor disclosed in Zhong et al, Bioorg. Med. Chem. Lett. (2011) 21(0:307-310; Wu et af, Inflamm. Res. (2003) 52(Suppl 2):Abs 137; Khojasteh et al, Xenobiotica (2008) 38(3):340-352; Pei et al., J. Med. Chem. (2001) 44(18):2913-2920; WO 2007/084882; or WO 02/059114; each of which is incorporated by reference in its entirety. Non-limiting examples include lifitegrast (SAR-1118; SHP-606; SPD-606; Xiidra; Sunesis Pharmaceuticals Inc.), BIRT-0377 (BIRT-2584; BIRT-2584 XX; BIRT-377; Boehringer Ingelheim International GmbH), CP151088 (Genentech/Roche), ICAM-2078 (Genentech/Roche), ICAM-850 (Genentech/Roche), RO-0276845 (Roche), RO-5182851 (Roche), RO-5184438 (Roche), RO-5200045 (Roche), LFA-451 (Novartis Pharma AG), LFA-703 (Novartis Pharma AG), LFA-878 (Novartis Pharma AG), XVA-143 (Novartis Pharma AG), IC-776 (ICOS Corp.), A-276594 (ICOS Corp.), A-286982 (ICOS Corp.), A-292949 (ICOS Corp.), A-295339 (ICOS Corp.), A-324920 (ICOS Corp.), and IC-52593 (ICOS Corp.).

In some embodiments, the integrin modulator is a phenylalanine derivative containing an optionally substituted pyridazinone ring, for example, see WO 2005/077914; Gong et al, J. Med. Chem, 49:3402-3411 (2006); Gong et al, Bioorg. Med. Chem. Lett., 18: 1331-1335 (2008).

In some embodiments, the integrin modulator is a compound disclosed in US 2005/0209232; U.S. Pat. No. 9,518,091; WO 2005/077914; WO 2005/077915; WO 2017/135471; WO 2017/135472; Dubree et al, J. Med. Chem. 45:3451-3457 (2002); Gong et al, J. Med. Chem, 49:3402-3411 (2006); Gong et al., Bioorg. Med. Chem. Lett., 18: 1331-1335 (2008); Sidduri et al., Bioorg. Med. Chem. Lett., 23: 1026-1031 (2013); or Xu et al, Bioorg. Med. Chem. Lett., 23:4370-4373 (2013), each of which is incorporated by reference in their entireties.

In some embodiments, the integrin modulator is a compound of Formula I, II, III, or IV, as described in WO 2019/246455, which is incorporated herein by reference in its entirety.

In certain embodiments, the integrin modulator is vedolizumab, AJM-300 (carotegrast methyl).

Immunosuppressant

In some embodiments, the chemical entity is an immunosuppressant. Non-limiting examples include methotrexate, azathioprine (Imuran®), cyclosporine, tacrolimus, mycophenolate mofetil (Cellcept®), and cyclophosphamide (Cytoxan®), systemic or oral corticosteroids, rapamycin, FK-506, and interferon-gamma. In certain embodiments, the immunosuppressant is cyclosporine.

Steroidal Anti-Inflammatory Agents

In some embodiments, the chemical entity is a steroidal anti-inflammatory agent. In certain embodiments, the chemical entity is a steroid such as corticosteroid, including glucocorticoids and mineralocorticoids. Non-limiting examples include aclometasone, aclometasone dipropionate, aldosterone, amcinonide, beclomethasone (e.g., beclomethasone 17), beclomethasone dipropionate, betamethasone, betamethasone dipropionate, betamethasone sodium phosphate, betamethasone valerate, budesonide (e.g., budesonide prednisone), clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, methylprednisolone, mometasone paramethasone, mometasone furoate monohydrate, prednicarbate, prednisolone, prednisone, tixocortol, tixocortol pivalate, triamcinolone, triamcinolone acetonide, triamcinolone alcohol and their respective pharmaceutically acceptable derivatives.

In certain embodiments, the chemical entity is selected from the group consisting of: beclomethasone 17, budesonide prednisone, prednisolone, and beclometasone dipropionate.

Non-Steroidal Anti-Inflammatory Agents

In some embodiments, the chemical entity is a non-steroidal anti-inflammatory agent. Non-limiting examples include aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., 5-ASA, acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicyl sulfuric acid, salsalate, sulfasalazine), thiazinecarboxamides (e.g., ampiroxicam, droxicam, isoxicam, lornoxicam, piroxicam, tenoxicam), ε-acetamidocaproic acid, s-adenosylmethionine, 3-amino-4-hydroxybutyric acid, amixetrine, bendazac, benzydamine, bucolome, difenpiramide, ditazol, emorfazone, fepradinol, guaiazulene, nabumetone, nimesulide, oxaceprol, paranyline, perisoxal, proquazone, superoxide dismutase, tenidap, and zileuton.

In certain embodiments, the chemical entity is a salicyclic acid derivative such as aminosalicylate (e.g., 5-aminosalicyclic acid).

Receptor-Interacting Protein Kinase 1 (RIPK1) Inhibitors

In some embodiments, the chemical entity is a receptor-interacting protein kinase 1 (RIPK1) inhibitor. Non-limiting examples include RIPA-56, Nec-1s (7-Cl—O-Nec1), Necrostatin-1, Necrostatin-2, GSK′ 547, GSK2982772, GSK481, and GSK3145095. In certain embodiments, the RIPK1 inhibitor is GSK2982772.

Receptor-Interacting Protein Kinase 2 (RIPK2) Inhibitors

In some embodiments the chemical entity is a receptor-interacting protein kinase 2 (RIPK2) inhibitor. In some embodiments, the RIPK2 inhibitor is a compound as disclosed in patent publication No. WO 2020139748 or patent publication No. WO 2020132384, each incorporated by reference herein in its entirety.

In some embodiments, the RIPK2 inhibitor can be e.g., Compound 8, or a compound as disclosed in He et al, ACS Med. Chem. Lett. 2017, 8, 1048-1053, incorporated by reference herein in its entirety.

EP4 Modulators

In some embodiments, the chemical entity is a Prostaglandin E2 receptor 4 (EP4) modulator. In certain embodiments, the chemical entity is an EP4 agonist.

Non-limiting examples include KAG-308; AGN205203; APS-999 Na; Cay10598 (19a); CP-044,519-02; EP4RAG; L-902688; Lubiprostone; ONO-4819CD; ONO AE1-329; ONO AE1-734; PGE1-OH; TCS 2510; γ-Lactam PGE analog 3; 11-Deoxy-PGE1; γ-Lactam PGE analog 2a; and γ-Lactam PGE analog 4.

In certain embodiments, the chemical entity is KAG-308.

Toll-Like Receptor (TLR) Modulators

In some embodiments, the chemical entity is a toll-like receptor (TLR) modulator. In some embodiments, the chemical entity is a TLR agonist. In some embodiments, the chemical entity is a TLR antagonist.

In some embodiments, the chemical entity is a TLR agonist. In some embodiments, the TLR agonist binds to and activates TLR2. In some embodiments, the TLR agonist binds to and activates TLR3. In some embodiments, the TLR agonist binds to and activates TLR4. In some embodiments, the TLR agonist binds to and activates TLR5. In some embodiments, the TLR agonist binds to and activates TLR6. In some embodiments, the TLR agonist binds to and activates TLR7. In some embodiments, the TLR agonist binds to and activates TLR8. In some embodiments, the TLR agonist binds to and activates TLR9. In some embodiments, the TLR agonist binds to and activates TLR10. In some embodiments, the TLR agonist binds to and activates TLR11. In some embodiments, the TLR agonist binds to and activates two or more (e.g., three, four, five, six, seven, eight, nine, ten, or eleven) TLRs (e.g., two or more of any of TLR1, TLR2, TLR3, TLR4, TLR5, TLR6, TLR7, TLR8, TLR9, TLR10, and TLR11 (in any combination)).

In some embodiments, the TLR agonist is a synthetic TLR agonist, a TLR mimic, or a small molecule. Non-limiting examples of TLR agonists are described in Bhardwaj et al., Cancer J. 16(4):382-391, 2010; Meyer et al., Exp. Opin. Investig. Drugs 17(7):1051-1065, 2008; Adams, Immunotherapy 1(6):949-964, 2009; Hennessy et al., Nat. Rev. Drug Discov. 9:293-307, 2010; and U.S. Pat. Nos. 7,498,409; 9,421,254; 8,409,813; 8,361,986; 8,795,678; 8,728,486; 8,636,979; 8,999,946; 9,359,360; 9,050,376; and 9,556,167; US 2014/0322271; US 2016/0206690; US 2009/0253622; US 2011/0135669; US 2011/0250175; US 2014/0220074; and US 2012/0219615; each incorporated by reference in its entirety herein.

In some embodiments, the TLR agonist is a peptide or a fusion protein (Huleatt et al., Vaccine 25: 763-775, 2007). In some embodiments, a TLR agonist specifically binds to and activates a single TLR (e.g., TLR4, TLR7, TLR8, or TLR9; Zhu et al., J. Clin. Invest. 120:607-616, 2010; Zhu et al., PNAS 105:16260-16265, 2008; Wang et al., J. Virol. 79(22):14355-14370, 2005). In some embodiments, the TLR agonist binds to and activates more than one TLR (e.g., Bacillus of Calmette-Guerin, Myobacterium bovis (BCG); Morton et al., Ann. Surg. 180(4):635-643, 1974; Mortoon et al., J. Clin. Oncol. ASCO Ann. Meeting Proceedings Part I 25(18 Suppl), 2007). In some embodiments, the TLR agonist is a TLR2/TLR6 agonist (e.g., Pam₂CSK4 or MALP-2 (Agnihotri et al., J. Med. Chem. 54: 8148-8160, 2011; Wu et al., J. Med. Chem. 53: 3198-3213, 2010)).

In some embodiments, the TLR agonist is administrated in combination with another composition (Dowling et al., Clin. Transl. Immunol. 5:e85, 2016). In some embodiments, the TLR agonist is an endogenous molecule released from dead cells (e.g., a heat shock protein (HSP) and mobility group box 1 (HMGB1); Asea et al., J. Biol. Chem. 277:15028-15034, 2002; Kepp et al., Cancer Metastasis 30: 61-69, 2011).

In some embodiments, the TLR agonist specifically binds and activates TLR3 (e.g., a synthetic agonist). Non-limiting examples of TLR agonists that bind and activate TLR3 are described in Nicodemus et al., Immunotherapy 2:137-140, 2010. In some embodiments, the TLR3 agonist is a synthetic double-stranded RNA (dsRNA) complex. For example, the TLR3 agonist can be a TLR3 mimic (e.g., polyadenosine-polyuridylic acid (poly A:U) (Veyrat et al., Oncotarget 7(50):82580-82593, 2016; Alizadeh et al., Iran J. Allergy Asthma Immunol. 12(2):161-167, 2013); rintatolimod (polyI: polyCU, Ampligen®) (Steinman et al., Nature 449: 419-426, 2007; Jasani et al., Vaccine 27(25-26):3401-3404, 2009; Strayer et al., PLoS One 7(3): e31334, 2012). In some embodiments, the TLR3 mimic is polyinosinic-polycytidylic acid stabilized with poly-L-lysine and carboxymethylcellulose (Poly-ICLC, Hiltonol®; Hawkins et al., J. Biol. Resp. Mod. 4:664-668, 1985; Butowski et al., J. Neurooncol. 91:175-182, 2009; Jeong et al., J. Neurochem. doi.10.1111, 2015). In some embodiments, the TLR3 agonist is RGC100 (Naumann et al., Clin. Dev. Immunol. 283649, 2013), IPH-3102 (Basith et al., Exp. Opin. Ther. Pat. 21: 927-944, 2011), or a variant thereof. In some embodiments, the TLR3 agonist is CQ-07001 (Clinquest). In some embodiments, the TLR3 agonist is Ampligen poly(I):poly(C12U) (Hemispherx Biopharma). In some embodiments, the TLR3 agonist is IPH-31XX (Innate Pharma). In some embodiments, the TLR3 agonist is MCT-465-dsRNA (MultiCell Technologies).

In some embodiments, the TLR agonist specifically binds to and activates TLR4 (Peri et al., J. Med. Chem. 57(9):3612-3622, 2014). In some embodiments, the TLR4 agonist is bacterial lipopolysaccharide (LPS) or a variant thereof. In some embodiments, the TLR4 agonist is monophosphoryl lipid A (MPL, MPLA, GLA, GLA-SE) (Ribi et al., J. Immunol. 6:567-572, 1984; Okemoto et al., J. Immunol. 176:1203-1208, 2006; Matzner et al., Int. J. Cancer 138:1754-1764, 2016; Cauwelaert et al., PLoS One 11(1):e0146372, 2016). In some embodiments, the TLR agonist is AS15 or AS02b (Brichard et al., Vaccine 25(Suppl. 2):B61-B71, 2007; Kruit et al., J. Clin. Oncol. 26(Suppl): Abstract 9065, 2008). In some embodiments, the TLR agonist is an aminoalkyl glucosaminide 4-phosphate (e.g., RC-529, Ribi.529, E6020) or a variant thereof (Baldridge et al., J. Endotoxin Res. 8:453-458, 2002; Morefield et al., Clin. Vaccine Immunol. 14: 1499-1504, 2007). In some embodiments, the TLR agonist is picibanil (OK-432) (Hazim et al., Med. J. Malaysia 71(6):328-330, 2016; Tian et al., Asian Pac J. Cancer Prev. 16(11):4537-4542, 2015; Rebuffini et al., Dent Rese. J. 9(Suppl. 2):S192-5196, 2012). In some embodiments, the TLR4 agonist is Spirulina complex polysaccharide (Kwanishi et al., Microbiol. Immunol. 57:63-73, 2013). In some embodiments, the TLR4 agonist is chitohexaose or a variant thereof (Panda et al., 8:e1002717, 2012; Barman et al., Cell Death Dis. 7:e2224, 2016). In some embodiments, the TLR4 agonist is E5564 (Eritoran) (Eisai). In some embodiments, the TLR4 agonist is CRX-675 or CRX-527 (GSK).

In some embodiments, the TLR agonist binds and activates TLR5. In some embodiments, the TLR5 agonist is flagellin or a variant thereof (e.g., entolimod (CBLB502)) (Yoon et al., Science 335: 859-864, 2012; Fukuzawa et al., J. Immunol. 187:3831-3839, 2011; Brackett et al., PNAS 113(7):E874-E883, 2015; Leigh et al., PLoS One 9(1):e85587, 2014; Hossain et al., Blood 120:255, 2012). In some embodiments, the TLR5 agonist is flagellin HuHa (Vaxinate) or flagellin HuM2e (Vaxinate).

In some embodiments, the TLR agonist binds and activates TLR7/8 (e.g., TLR7 agonist, TLR8 agonist, or a TLR7 and TLR8 agonist). In some embodiments, the TLR7/8 agonist is ANA975 (isotorabine) (Anadys/Novartis), ANA773 (Anadys/Novartis), In some embodiments, the TLR7/8 agonist is an imidazoquinoline or a variant thereof (e.g., imiquimod (Aldara™; Kaspari et al., British J. Dermatology 147: 757-759, 2002; Smorlesi et al., Gene Therapy 12: 1324-133, 2005; Prins et al., J. Immunol. 176: 157-164, 2006; Shackleton et al., Cancer Immun. 4:9, 2004; Green et al., Br. J. Dermatol. 156(2):337-345, 2007; Geisse et al., Am. Acad. Dermatol. 50(5):722-733, 2004; Wolf et al., Arch. Dermatol. 139(3):273-276, 2003), resiquimod (R848; Hemmi et al., Nat. Immunol. 3:196-200, 2002; Jurk et al., Nat. Immunol. 3:49, 2002; Rook et al., Blood 126(12):1452-1461, 2015; Dovedi et al., Blood 121: 251-259, 2013). In some embodiments, the TLR agonist is a synthetic imiadzoquinoline mimicking viral single stranded RNA (ssRNA) (852A) or a variant thereof (Dudek et al., Clin. Cancer Res. 13(23):7119-7125, 2007; Dummer et al., Clin. Cancer Res. 14(3):856-864, 2008; Weigel et al., Am. J. Hematol. 87(10):953-956, 2012; Geller et al., Cancer Immunol. Immunother. 59(12):1877-1884, 2010; Inglefield et al., J. Interferon Cytokine Res. 28(4):253-263, 2008). In some embodiments, the TLR agonist is a small molecule. In some embodiments, the small molecule mimics viral ssRNA (e.g., motolimod (VTX-2337)) or a variant thereof (Dietsch et al., Clin. Cancer Res. 21(24):5445-5452, 2015; Northfelt et al., Clin. Cancer Res. 20(14):3683-3691, 2014; Lu et al., Clin. Cancer Res. 18(2):499-509, 2012). In some embodiments, the small molecule is GS-9620 or a variant thereof (Bam et al., Antimicrob Agents Chemother. 61(1):e01369, 2016; Rebbapragada et al., PLoS One 11(1):e0146835, 2016; Gane et al., J. Hepatol. 63(2): 320-328, 2015; Fosdick et al., J. Med. Chem. 56(18):7324-7333, 2013). In some embodiments, the small molecule is SC1 (Wiedemann et al., Oncoimmunology 5(7):e1189051, 2016; Hamm et al., J. Immunol. 6(4):257-265, 2009). In some embodiments, the small molecule is gardiquimod (Ma et al., Cell. Mol. Immunol. 7:381-388, 2010; Hjelm et al., Hum. Vaccin. Immunother. 10(2): 410-416, 2014; Buitendijk et al., AIDS Res. Hum. Retroviruses 29(6):907-918, 2013), CL075 (Philbin et al., J. Allergy Clin. Immunol. 130:195-204, 2012; Dowling et al., PLoS One 8(3): e58164, 2013), CL097 (Gorden et al., J. Immunol. 174:1259-1268, 2005; Gorski et al., Int. Immunol. 18:1115, 2006; Levy et al., Blood 108:1284-1289, 2006; Wille-Reece et al., J. Exp. Med. 203: 1249-1258, 2006), loxoribine (Pope et al., Cell Immunol. 162:333, 1995; Heil et al., Eur. J. Immunol. 33:2987-2997, 2003; Lee et al., PNAS 100:6646-6651, 2003), or VTX-294 (Dowling et al., PLoS One 8(3):e58164, 2013). In some embodiments, the TLR7/8 agonist is IMO-9200. In some embodiments, the TLR7 agonist is IPH-32XX (Innate Pharma).

In some embodiments, the TLR agonist binds and activates TLR9. In some embodiments, the TLR9 agonist is a synthetic oligonucleotide. In some embodiments, the synthetic oligonucleotide contains unmethylated CpG dinucleotide (CpG-ODN). Additional non-limiting examples of TLR9 agonists include cobitolimod, PF-3512676, IMO-2055 (EMD1201801), DIMS0150; CpG7909 (Vaximmune); IMO-9200; AVE0675 (Coley, Sanofi Aventis); or Amplivax (Idera).

In some embodiments, the TLR agonist is a bacterial or viral component. In some embodiments, the TLR agonist is derived from the cell wall Mycobacterium bovis (BCG). In some embodiments, the Mycobacterium bovis cell wall component is a TLR2 and/or TLR4 agonist (e.g., SMP105 (Murata et al., Cancer Sci. 99:1435-1440, 2008; Miyauchi et al., Drug Discov. Ther. 6: 218-225, 2013; Tsuji et al., Infect Immun. 68: 6883-6890, 2000; Smith et al., Cancer Immunol. Immunother. 63(8):787-796, 2014). Additional examples of TLR agonists are known in the art.

In some embodiments, the chemical entity is a TLR antagonist. In certain embodiments, the TLR antagonist decreases the binding of a TLR agonist to TLR4 or TLR9 expressed in a mammalian cell (e.g., a human cell). In some embodiments, any of the compositions or methods described herein can include a TLR antagonist. For example, a TLR antagonist can be a TLR4 antagonist. In other examples, a TLR antagonist is a TLR9 antagonist. Non-limiting examples of TLR antagonists are described in Fukata et al., Mucosal Immunity 6:451-463, 2013.

Non-limiting examples of TLR4 antagonists include JKB-122; 1A6; CRX-526; eritoran tetrasodium (E5564), small heat shock protein B8 (HSP22), CRX-527, E5564, IAXO-102, AG-411, CRX-52624, and E5531. In certain embodiments, the TLR4 antagonist is JKB-122.

Non-limiting examples of TLR9 antagonists include adenoviral oligodeoxynucleotides (AV-ODN) (Obermeier et al., Gastroenterology 129:913-927, 2005); ODN 2088; ODN 4084-F; ODN INH-1; ODN INH-18; ODN TTAGGG (A151); and G-ODN (each commercially available from InvivoGen). In some embodiments, the TLR9 antagonist is CpG-ODN c41. In some embodiments, the TLR9 antagonist is COV08-0064; ODN 1585, ODN 1826, ODN 2395, and ODN 2088; IMO-8400; IRS869; IMO-3100; TTAGGG; and CpG ODN.

In some embodiments, the TLR modulator is BL-7040. In some embodiments, the TLR modulator is EN-101. In some embodiments, the TLR modulator is Monarsen.

In some embodiments, the TLR modulator is JKB-122 or cobitolimod.

Smad7 Modulators

In some embodiments, the chemical entity is an Smad7 modulator. In certain embodiments, the chemical entity is an Smad7 antisense oligonucleotide. As a non-limiting example, the chemical entity can be mongersen.

Phosphodiesterase 4 (PDE4) Modulators

In some embodiments, the chemical entity is a phosphodiesterase 4 (PDE4) modulator. In certain embodiments, the chemical entity is a PDE4 inhibitor.

In some embodiments, the PDE4 modulator is a small molecule, an antibody, a peptide fragment, or a nucleic acid. Non-limiting examples of small molecules, antibodies, peptide fragments, and nucleic acids that are PDE modulators include those described in WO 2019/147824 which is incorporated herein by reference in its entirety.

Non-limiting examples of PDE4 modulators include: apremilast, CC-1088, tetomilast, KF-19514, PF-06266047, SKF-107806, PDB-093, tolafentrine (BY-4070), TAK-648, CH-928, CH-673, CH-422, ABI-4 (18F-PF-06445974), roflumilast N-oxide, BYK-321084, WAY-127093B, NCS-613, SDZ-ISQ-844, Ro-20-1724, Hemay-005, filaminast (PDA-641), LASSBio-596, ASP-3258, TAS-203, lotamilast, GPD-1116, cipamfylline, ZL-N-91, CDP-840, GSK-356278, cilomilast, MNP-001, KF-66490, cilomilast, OCID-2987, roflumilast, AN-2898, CBS-3595, ASP-9831, E-4021, piclamilast, CD-160130, GSK-256066, 4AZA-PDE4, YM-393059, revamilast, crisaborole, MK-0952, ibudilast, GP-0203, ELB-526, theophylline, CHF-6001, elbimilast, AWD-12-281, ibudilast, OS-0217, oglemilast, R-1627, ND-1510, ND-1251, WAY-122331, GRC-3566, tofimilast, BAY-61-9987, rolipram, MEM-1414, CGH-2466, RPL-554, CT-5357, etazolate, Qrg-30029, Z-15370, Org-20241, arofylline, KW-4490, HT-0712, CT-2450, SDZ-PDI-747, AP-0679, Sch-351591, TA-7906, HMR-1571, lirimilast, daxalipram, roflumilast, NVP-ABE-171, CC-10036, RPR-117658, AWD-12-281, 256066, arofylline derivatives, RPR-132294, KC-404, CI-1018, YM-976, XT-611, losartan derivatives, DWP-205, WAY-126120, YM-58997, CP-293321, V-11294A, CH-3697, CP-353164, atizoram, D-4418, RPR-114597, IC-197, CP-220629, ZL-n-91, D-22888, and GW-3600.

Non-limiting examples of PDE4 modulators (e.g., inhibitors) also include: Apremilast (CC-10004; CC-110004; CDC-104; Otezla®; lead selCID (2); selCID); CC-1088 (CC-1088; CC-5048; CC-801; CDC-801; lead SelCID (1)); Tetomilast (OPC-6535); KF-19514; PF-06266047; SKF-107806; PDB-093; Tolafentrine (BY-4070); TAK-648; CH-928; CH-673; CH-422; ABI-4 (18F-PF-06445974; Fluorine-18-PF-06445974); roflumilast; Roflumilast N-oxide (APTA-2217; B9302-107; BY-217; BYK-20860; Daliresp®; Dalveza; Daxas®; Libertek; Xevex; roflumist); NVP-ABE-171; BYK-321084; WAY-127093B; NCS-613; SDZ-ISQ-844; GS-5759; Ro-20-1724; Hemay-005; KCA-1490; TVX-2706; Nitraquazone; Filaminast (PDA-641; WAY-PDA-641); LASSBio-596; ASP-3258; TAS-203; AN-2889; AN-5322; AN-6414; AN-6415; Iotamilast (E-6005; RVT-501); GPD-1116; Cipamfylline (BRL-61063; HEP-688); MNP-001; MS-23; MSP-001; K-34; KF-66490; AL-38583 (cilomast); ZL-N-91; Almirall; CDP-840; GSK-356728; Cilomilast (Ariflo; SB-207499); OCID-2987; AN-2898; CBS-3595; ASP-9831 (ASP9831); E-4021 (4-Piperidinecarboxylic acid, 1-[4-[(1,3-benzodioxol-5-ylmethyl)amino]-6-chloro-2-quinazolinyl]); Piclamilast (RP-73401; RPR-73401); CD-160130; GSK-256066 (256066); 4AZA-PDE4; YM-393059; Revamilast (GRC-4039); AN-2728 (PF-06930164; crisaborole (Eucrisa™)); MK-0952 (MK-952); Ibudilast (AV-411; MN-166; KC-404); GP-0203; ELB-526; Theophylline (Teonova); CHF-6001 (CHF-5480); Elbimilast (AWD-12-353; ELB-353; ronomilast); AWD-12-281 (842470); OS-0217; Oglemilast (GRC-3886); R-1627; ND-1510; ND-1251; WAY-122331; GRC-3566; Tofimilast (CP-325366); BAY-61-9987; Rolipram (ME-3167; ZK-62711); MEM-1414 (R-1533); Adenosine A3 antagonists (CGH-2466); RPL-554 (RPL-565; VMX-554; VMX-565; VRP-554; trequinsin analog); CT-5357; Etazolate (EHT-0202; SQ-20009; etazolate hydrochloride); Z-15370 (Z-15370A); Org-30029; Org-20241; Arofylline (LAS-31025); Arofylline derivatives; KW-4490; HT-0712 (IPL-455903); HT-0712; IPL-455903; CT-2450; CT-2820; CT-3883; CT-5210; L-454560; L-787258; L-791943; L-826141; L-869298; MK-0359; OX-914 (BLX-028914; BLX-914; IPL-4088; IPL-4182; IPL-4722); SDZ-PDI-747; AP-0679; Sch-351591 (D-4396; Sch-365351); TA-7906 (T-2585; TA-7906); HMR-1571; Lirimilast (BAY-19-8004); Daxalipram (Mesopram; SH-636; ZK-117137); SelCIs (CC-10036; CC-10083; CC-110007; CC-110036; CC-110037; CC-110038; CC-110049; CC-110052; CC-110083; CC-11069; CC-111050; CC-13039; CC-14046; CC-17034; CC-17035; CC-17075; CC-17085; CC-18062; CC-7075); RPR-117658; AWD-12-281 (842470; AWD-12-343; GW842470X); 256066 (GSK-256066; SB-207499); RPR-132294 (RPR-132703); CI-1018; CI-1044; PD-168787; PD-189659; PD-190036; PD-190749; YM-976; XT-611; Losartan derivatives; DWP-205 derivatives (DWP-205297); WAY-126120; YM-58997; CP-293321; V-11294A; CH-3697; CP-353164; Atizoram (CP-80633); D-4418; RPR-114597; IC-197; IC-246; IC-247; IC-485; IC-86518; IC-86518/IC-86521; IC-86521; CP-220629; ZL-n-91; D-22888 (AWD-12-232); GW-3600; GSK356278; TPI 1100; BPN14770; and MK-0873. See, e.g., Schafter et al. (2014) Cellular Signaling 26(9):2016-2029); Gurney et al. (2011) Handb Exp Pharmacol 204:167-192; Spadaccini et al. (2017) Intl J Mol Sciences 18:1276; Bickston et al. (2012) Expert Opinion Invest Drugs 21:12, 1845-1849; Keshavarzian et al. (2007) Expert Opinion Invest Drugs 16:9, 1489-1506.

In certain embodiments, the PDE4 modulator is apremilast.

Tumor Progression Locus 2 (TPL2) Inhibitors

In some embodiments, the chemical entity is a tumor progression locus 2 (TPL2) inhibitor. In certain embodiments, the chemical entity is GS-4875.

Tyrosine Kinase 2 (TYK2) Inhibitors and TEC Kinase Inhibitors

In some embodiments, the chemical entity is a tyrosine kinase 2 (TYK2) inhibitor (e.g., Masse et al., J. Immunol. 194(1):67, 2015; Menet, Pharm. Pat. Anal. 3(4):449-466, 2014; Liang et al., Euro. J. Med. Chem. 67: 175-187, 2013; Jang et al., Bioorg. Med. Chem. Lett. 25(18):3947-3952, 2015); U.S. Pat. Nos. 9,296,725 and 9,309,240; US 2013/0231340; and US 2016/0251376). In some embodiments, the TYK2 inhibitor is Ndi-031301 (Akahane et al., Blood 128:1596, 2016); BMS-986165 (Gillooly et al., 2016 ACR/ARHP Annual Meeting, Abstract 11L, 2016); SAR-20347 (Works et al., J. Immunol. 193(7):3278-3287, 2014); tyrphostin A1 (Ishizaki et al., Int. Immunol. 26(5):257-267, 2014); a triazolopyridine (US 2013/0143915); PF-04965842; PF-06651600; PF-06700841; PF-06826647; or a variant thereof.

In certain embodiments, the TYK2 inhibitor is BMS-986165, PF-06700841, or PF-06826647.

In some embodiments, the chemical entity is a TEC kinase inhibitor, such as PF-06651600.

Phosphatidylcholine

In some embodiments, the chemical entity is phosphatidylcholine, e.g., LT-02.

TIP60 Inhibitor

In some embodiments, the chemical entity is a TIP60 inhibitor (see, e.g., U.S. Patent Application Publication No. 2012/0202848).

In some embodiments, the chemical entity is selected from the group consisting of diphenoxylate/atropine, loperamide, infliximab, 6-mercaptopurine, AbGn-168H, ABX464, ABT-494, adalimumab, AJM300, alicaforsen, AMG139, anrukinzumab, apremilast, ATR-107 (PF0530900), autologous CD34-selected peripheral blood stem cells transplant, azathioprine, bertilimumab, BI 655066, BMS-936557, certolizumab pegol (Cimzia®), cobitolimod, CP-690,550, CT-P13, DIMS0150, E6007, E6011, etrasimod, etrolizumab, fecal microbial transplantation, figlotinib, fingolimod, firategrast (SB-683699) (formerly T-0047), GED0301, GLPG0634, GLPG0974, guselkumab, golimumab, GSK1399686, HMPL-004 (Andrographis paniculata extract), IMU-838, Interleukin 2 (IL-2), laquinimod, masitinib (AB1010), matrix metalloproteinase 9 (MMP 9) inhibitors (e.g., GS-5745), MEDI2070, mesalamine, methotrexate, mirikizumab (LY3074828), natalizumab, NNC 0142-0000-0002, NNC0114-0006, ozanimod, peficitinib (JNJ-54781532), PF-00547659, PF-04236921, PF-06687234, QAX576, RHB-104, rifaximin, risankizumab, RPC1063, SB012, SHP647, sulfasalazine, TD-1473, thalidomide, tildrakizumab (MK 3222), TJ301, TNF-Kinoid®, tofacitinib, tralokinumab, TRK-170, upadacitinib, ustekinumab, UTTR1147A, V565, vatelizumab, VB-201, vedolizumab, and vidofludimus.

Emoxypine and/or Glutamate 2b (Glut2B or GluN2B) Receptor Antagonists

In some embodiments the chemical entity is emoxypine and/or an antagonist of Glutamate 2b (Glut2B or GluN2B) receptor. In some embodiments, the Glut2B or GluN2B antagonist is selected from a group consisting of ifenprodil, radiprodil, traxoprodil, rislenmdaz, eliprodil, Ro-25-6981, BMT-108908, EVT-101, CP101-606, MK-0657, EVT-103, and AZD 6765 (Annual Reports in Medicinal Chemistry (2012) Volume 47: 94-103), and/or a compound as disclosed in patent publication No. WO 2021003553, incorporated by reference herein in its entirety.

Pantetheinase Inhibitors

In some embodiments the chemical entity is a pantetheinase inhibitor. In some embodiments, the pantetheinase is Vanin-1 (VNN1). In some embodiments, the Vanin-1 inhibitor is a compound as disclosed in patent publication No. WO 2020102575 or WO 2020114943 or WO 2018228934 or U.S. Ser. No. 10/364,255 B2, each incorporated by reference herein in its entirety.

Bile Salt Hydrolase Modulators

In some embodiments the chemical entity is a bile salt hydrolase (BSH) modulator. In some embodiments, the BSH modulator is a compound as disclosed in patent publication No. WO 2020231776, incorporated by reference herein in its entirety.

Aryl Hydrocarbon Receptor (AhR) Agonists

In some embodiments the chemical entity is an aryl hydrocarbon receptor (AhR) agonist. In some embodiments, the AhR agonist is a compound as disclosed in patent publication No. WO 20200227151, incorporated by reference herein in its entirety.

In some embodiments, the AhR agonist can be indole-3 carbinol or diindolylmethane as disclosed in Neavin et al., Int. J. Mol. Sci. 2018, 19, 3851, incorporated by reference herein in its entirety.

In some embodiments, the AhR agonist can be NPD-0414-2 or NPD-0414-24 as disclosed in Marafini et al., Front. Pharmacol. 10:380. doi: 10.3389/fphar.2019.00380, incorporated by reference herein in its entirety.

In some embodiments, the AhR agonist can be PY109 or PY108 or a compound as disclosed in Chen et al., Sci. Adv. 6, eaay8230 (2020), incorporated by reference herein in its entirety.

Prolyl Hydroxylases Inhibitors

In some embodiments the chemical entity is a prolyl hydroxylases (PHDs) inhibitors. In some embodiments, the PHD inhibitor is a compound as disclosed in patent publication No. WO 2020054788, incorporated by reference herein in its entirety.

In some embodiments, the PHD inhibitor is AKB-4924 or a compound as disclosed in patent publication No. WO 2020054825, incorporated by reference herein in its entirety.

GABAA Receptor Agonist

In some embodiments the chemical entity is a selective GABAA receptor agonist (δ-subunit containing GABAA receptors). In some embodiments, the GABAA receptor agonist is gaboxadol as disclosed in patent publication No. WO 2020041574, incorporated by reference herein in its entirety.

KEAP1-Nrf2 Inhibitors

In some embodiments the chemical entity is a KEAP1-Nrf2 inhibitor. In some embodiments, the KEAP1-Nrf2 inhibitor is a compound as disclosed in patent publication No. WO 2020041169 A2, incorporated by reference herein in its entirety.

In some embodiments, the KEAP1-Nrf2 inhibitor is a compound as disclosed in patent publication No. WO 2020150446, incorporated by reference herein in its entirety.

### Human Histamine 4 Receptor Inhibitors

In some embodiments the chemical entity is a human histamine 4 receptor (hH4R) inhibitor. In some embodiments, the hH4R inhibitor is a compound as disclosed in patent publication No. WO 2019231270, incorporated by reference herein in its entirety.

Peptidylarginine Deiminase Inhibitors

In some embodiments the chemical entity is a peptidylarginine deiminase (PAD4) inhibitor. In some embodiments, the PAD4 inhibitor is a compound as disclosed in patent publication No. WO 2019161803 or patent publication No. WO 2016185279 or patent publication No. WO 2019077631 or patent publication No. WO 2019058393, each incorporated by reference herein in its entirety.

Tumor Necrosis Factor-α Inhibitors

In some embodiments the chemical entity is a tumor necrosis factor-α (TNF-α) inhibitor. In some embodiments, the TNF-α inhibitor is a compound as disclosed in patent publication No. WO 2019146773 or patent publication No. WO 2015011331, each incorporated by reference herein in its entirety.

MEK/ERK Inhibitors

In some embodiments the chemical entity is a MEK/ERK inhibitor. In some embodiments, the MEK/ERK inhibitor is mebendazole as disclosed in patent publication No. WO 2019121996 in incorporated by reference herein in its entirety.

Interleukin-1 Receptor-Associated Kinase 4 Inhibitors

In some embodiments the chemical entity is an interleukin-1 receptor-associated kinase 4 (IRAK4) inhibitor. In some embodiments, the IRAK4 inhibitor is a compound as disclosed in patent publication No. WO 2017207386 or patent publication No. WO 2015193846, each incorporated by reference herein in its entirety.

In some embodiments, the IRAK4 inhibitor is a bicyclic heterocyclic compound as disclosed in patent publication No. WO 2013042137 or patent publication No. WO 2014058685, each incorporated by reference in its entirety.

In some embodiments, the IRAK4 inhibitor is a compound as disclosed in Bahia et al., Cell Signal. 2015 June; 27(6):1039-55. doi: 10.1016/j.cellsig.2015.02.025), incorporated by reference herein in its entirety.

In some embodiments, the IRAK4 inhibitor is a compound as disclosed in Wang et al., Curr Top Med Chem, 2009, 9(8), 724-737. https://doi.org/10.2174/156802609789044407, incorporated by reference in entirely.

Protein Kinase C Inhibitors

In some embodiments the chemical entity is a protein kinase C (PKC) inhibitor. In some embodiments, the PKC inhibitor is a compound as disclosed in patent publication No. WO 2017087318 incorporated by reference herein in its entirety.

CCR9 Chemokine Receptor Inhibitors

In some embodiments the chemical entity is a CCR9 chemokine receptor inhibitor. In some embodiments, the CCR9 inhibitor is CCX507 as disclosed in patent publication No. WO 2016057424, also as disclosed in Walter et al., Immunology Letters, 151, (2013), 44-47, each incorporated by reference herein in its entirety.

In some embodiments, the CCR9 inhibitor is vercirnon (also known as Traficet-EN or CCX282), as disclosed in Löwenberg et al., Curr Gastroenterol Rep (2015) 17: 21 incorporated by reference herein in its entirety.

In some embodiments, the CCR9 inhibitor is a compound as disclosed in Zhang et al., Bioorg. Med. Chem. Lett. 25 (2015) 3661-3664, incorporated by reference herein in its entirety.

Retinoid-Related Orphan Receptor Inhibitors

In some embodiments the chemical entity is a Retinoid-related Orphan Receptor gamma (RORγt) inhibitor. In some embodiments, the RORγt inhibitor is a compound as disclosed in patent publication No. WO 2016039408 or patent publication No. WO 2015090507 or issued U.S. Pat. No. 8,389,739 B1, each incorporated by reference herein in its entirety.

In some embodiments, the RORγt inhibitor can be B1119 as disclosed in Bassolas-Molina et al., Frontiers Immunol. 9, 2307, (2018), doi:10.3389/fimmu.2018.02307, incorporated by reference herein in its entirety.

Nuclear Factor-Kappa B Activation Inhibitors

In some embodiments the chemical entity is a nuclear factor-kappa B (NF-κB) activation inhibitor. In some embodiments, the NF-κB inhibitor is a compound as disclosed in patent publication No. WO 2015028976, incorporated by reference herein in its entirety.

In some embodiments, the NF-κB inhibitor is 3-[(dodecylthiocarbonyl)-methyl]-glutarimide (DTCM-G) or dehydroxymethylepoxyquinomicin (DHMEQ), as disclosed in El-Salhy, et al., International Journal of Molecular Medicine 37, no. 6 (2016): 1457-1464. https://doi.org/10.3892/ijmm.2016.2560; Funakoshi et al., or Journal of Crohn's and Colitis, 6 (2), 2012, 215-225. https://doi.org/10.1016/j.crohns.2011.08.011, each incorporated by reference herein in its entirety.

In some embodiments, the NF-κB inhibitor is a compound as disclosed in Zaidi et al. Front. Pediatr. 6:317. doi: 10.3389/fped.2018.00317, incorporated by reference herein in its entirety.

In some embodiments, the NF-κB inhibitor is a fumarate compound as disclosed in patent publication No. WO 2016127186 or patent publication No. WO 2016133832, each incorporated by reference herein in its entirety.

G Protein-Coupled Bile Acid Receptor 1 (GPBAR1; TGR5) Agonists

In some embodiments the chemical entity is a G protein-coupled bile acid receptor 1 (GPBAR1; TGR5) agonist. In some embodiments, the TGR5 agonist is a compound as disclosed in patent publication No. WO 2017106818, incorporated by reference herein in its entirety.

Peroxisome Proliferator-Activated Receptor Gamma (PPARγ) Modulators

In some embodiments the chemical entity is a PPARγ modulator. In some embodiments, the PPARγ modulator is 3-(4-aminophenyl)-2-methoxypropionic acid (GED-0507-34L) as disclosed in patent publication No. WO 2017093444, incorporated by reference herein in its entirety. In some embodiments, the PPARγ modulator is a compound as disclosed in Decara et al., Front. Pharmacol. 11:730. doi: 10.3389/fphar.2020.00730, incorporated by reference herein in its entirety.

5-Aminolevulinic Acid

In some embodiments the chemical entity is 5-aminolevulinic acid as disclosed in patent publication No. WO 2020221827, incorporated by reference herein in its entirety.

In some embodiments, the chemical entity is a compound described in the following references, each of which is incorporated by reference herein in its entirety:
Klepsch et al., Front Immunol. 2019; 10: 1070. doi:10.3389/fimmu.2019.01070;
https://doi.org/10.1371/journal.pone.0155771;
Schwaid et al., J Med. Chem. 2021; 64 (1):101-122. doi:10.1021/acs.jmedchem.0c01307;
Algieri et al, Mediators of Inflammation, vol. 2015, Article
ID 179616, 14 pages, 2015. https://doi.org/10.1155/2015/179616;
Hazel et al., Therapeutic Advances in Chronic Disease. January 2020.
doi:10.1177/2040622319899297
Zahid et al., Front Immunol. 2019; 10:2538. Published 2019 Oct. 25.
doi:10.3389/fimmu.2019.02538
D'Amico et al; Current Opinion in Pharmacology 2020, 55:141-150.
https://doi.org/10.1016/j.coph.2020.10.015;
Bai et al., European Journal of Medicinal Chemistry 185 (2020) 111805;
Kobayashi et al. Nat Rev Dis Primers 6, 74 (2020). https://doi.org/10.1038/s41572-020-0205-x;
Currie et al., Bioorganic & Medicinal Chemistry Letters, 29(16), 2019, 2034-2041,
https://doi.org/10.1016/j.bmcl.2019.06.042.

In some embodiments, the checkpoint inhibitor is a compound described in the following references, each of which is incorporated by reference herein in its entirety:
Burugu et al., Seminars in Cancer Biology 52 (2018) 39-52;
Friedlaender et al., ESMO Open 2019; 4:e000497. doi:10.1136/esmoopen-2019-000497;
Long, et al., Genes Cancer. 2018 May; 9(5-6):176-189. doi: 10.18632/genesandcancer.180.

### Methods of Treatment

In some embodiments, iatrogenic autoimmune colitis is colitis induced by one or more chemotherapeutic agents. In some embodiments, iatrogenic autoimmune colitis is colitis induced by treatment with adoptive cell therapy. In some embodiments, iatrogenic autoimmune colitis is colitis associated by one or more alloimmune diseases (such as graft-vs-host disease, e.g., acute graft vs. host disease and chronic graft vs. host disease). In other embodiments, the iatrogenic autoimmune colitis can result from Clostridium difficile infection, which is among the leading cause of nosocomial diarrhea and colitis in the industrialized world and typically occurs in subjects taking broad spectrum antibiotics.

In certain embodiments, the autoimmune colitis is induced by one or more chemotherapeutic agents, e.g., a chemotherapeutic immunomodulator, e.g., an immune checkpoint inhibitor. In certain of these embodiments, the immune checkpoint inhibitor targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (DO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MEW class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155; e.g., CTLA-4 or PD1 or PD-L1). See, e.g., Postow, M. J. Clin. Oncol. 2015, 33, 1.

In certain of these embodiments, the immune checkpoint inhibitor is selected from the group consisting of: Urelumab, PF-05082566, MEDI6469, TRX518, Varlilumab, CP-870893, Pembrolizumab (PD1), Nivolumab (PD1), Atezolizumab (formerly MPDL3280A) (PDL1), MEDI4736 (PD-L1), Avelumab (PD-L1), PDR001 (PD1), BMS-986016, MGA271, Lirilumab, IPH2201, Emactuzumab, INCB024360, Galunisertib, Ulocuplumab, BKT140, Bavituximab, CC-90002, Bevacizumab, and MNRP1685A, and MGA271.

Non-limiting examples of checkpoint inhibitors also include ipilimumab, tremelimumab, nivolumab, pidilizumab, MPDL3208A, MEDI4736, MSB0010718C, BMS-936559, BMS-956559, BMS-935559 (MDX-1105), AMP-224, and pembrolizumab.

In certain of these embodiments, the immune checkpoint inhibitor targets CTLA-4, e.g., an antibody, e.g., ipilimumab or tremelimumab. In certain embodiments, immune checkpoint inhibitor is ipilimumab.

In certain of these embodiments, the immune checkpoint inhibitor targets PD1 or PD-L1, e.g., nivolumab, lambroizumab, or BMS-936559.

In some embodiments, the subject is a human.

In some embodiments, the subject is undergoing or has undergone treatment for cancer. Non-limiting examples of cancer include: multiple myeloma, leukemias (HTLV-1 dependent, erythroleukemia, acute myelogenous leukemia (AML), chronic myelogenous leukemia (CIVIL), and large granular lymphocyte leukemia (LGL), lymphomas (EBV-related/Burkitt's, mycosis fungoides, cutaneous T-cell lymphoma, non-Hodgkins lymphoma (NHL), anaplastic large-cell lymphoma (ALCL), breast cancers, triple-negative breast cancers, head and neck cancers, melanoma, ovarian cancers, lung cancers, pancreatic cancers, prostate cancers, sarcomas, osteosarcoma, Kaposi's sarcoma, Ewing's sarcoma, hepatocellular cancers, glioma, neuroblastoma, astrocytoma, colorectal cancers, Wilm's tumors, renal cancers, bladder cancers, endometrial cancers, cervical cancers, esophageal cancers, cutaneous squamous cell cancers, basal cell cancers, and metastatic cancers.

In some embodiments, the subject is undergoing or has undergone treatment for cancer selected from the group consisting of: skin cancers (e.g., malignant melanoma (MM), Merkel cell carcinoma (MCC), cutaneous squamous cell carcinoma (CSCC)); lung cancers (e.g., non-small cell lung cancer (NSCLC) or small cell lung cancer (SCLC)); liver cancers (e.g., hepatocellular carcinoma (HCC)); MSI-H or dMMR cancers (e.g., any MSI-H or dMMR cancers, or colorectal cancer (CRC)); genitourinary cancers (e.g., metastatic urothelial carcinoma (mUC) or cervical cancer); head and neck cancers (head and neck squamous cell carcinoma (SCCHN)); gastric cancers (e.g., gastric or gastroesophageal junction (GEJ) adenocarcinoma); kidney cancers (e.g., renal cell carcinoma (RCC)); hematological cancers (e.g., classical Hodgkin Lymphoma (CHL) or primary mediastinal large B-cell lymphoma (PMBCL)); and breast cancers (e.g., triple-negative breast cancer (TNBC)).

In certain of these embodiments, the cancer is selected from the group consisting of: malignant melanoma (MM), Merkel cell carcinoma (MCC), cutaneous squamous cell carcinoma (CSCC), non-small cell lung cancer (NSCLC), small cell lung cancer (SCLC), hepatocellular carcinoma (HCC), MSI-H or dMMR cancers, colorectal cancer (CRC), metastatic urothelial carcinoma (mUC) or cervical cancer, head and neck squamous cell carcinoma (SCCHN), gastric or gastroesophageal junction (GEJ) adenocarcinoma, renal cell carcinoma (RCC), classical Hodgkin Lymphoma (CHL), primary mediastinal large B-cell lymphoma (PMBCL); and triple-negative breast cancer (TNBC).

In some embodiments, the treatment for cancer comprises administering to the subject an immune checkpoint inhibitor. In certain of these embodiments, the immune checkpoint inhibitor targets CTLA-4. As a non-limiting example, the immune checkpoint inhibitor can be ipilimumab or tremelimumab.

In certain embodiments (when the subject is undergoing or has undergone treatment for cancer; and the treatment for cancer comprises administering to the subject an immune checkpoint inhibitor), the immune checkpoint inhibitor targets PD1 or PD-L1. As a non-limiting example of the foregoing embodiments, the immune checkpoint inhibitor can be selected from the group consisting of: pembrolizumab, nivolumab, atezolizumab, avelumab, lambroizumab, cemiplimab, and BMS-936559.

In certain embodiments (when the subject is undergoing or has undergone treatment for cancer; and the treatment for cancer comprises administering to the subject an immune checkpoint inhibitor), the immune checkpoint inhibitor targets CD274 (e.g., the immune checkpoint inhibitor is durvalumab).

In certain embodiments, the subject is undergoing or has undergone treatment for malignant melanoma (e.g., metastatic melanoma); and the treatment comprises administering to the subject one or more of ipilimumab, nivolumab, or pembrolizumab.

In certain embodiments, the subject is undergoing or has undergone treatment for Merkel call carcinoma; and the treatment comprises administering to the subject one or more of avelumab or pembrolizumab.

In certain embodiments, the subject is undergoing or has undergone treatment for cutaneous squamous cell carcinoma (CSCC); and the treatment comprises administering to the subject cemiplimab.

In certain embodiments, the subject is undergoing or has undergone treatment for non-small cell lung cancer (NSCLC); and the treatment comprises administering to the subject one or more of pembrolizumab, nivolumab, atezolizumab, or durvalumab.

In certain embodiments, the subject is undergoing or has undergone treatment for small cell lung cancer (SCLC); and the treatment comprises administering to the subject nivolumab.

In certain embodiments, the subject is undergoing or has undergone treatment for hepatocellular carcinoma; and the treatment comprises administering to the subject one or more of pembrolizumab or nivolumab.

In certain embodiments, the subject is undergoing or has undergone treatment for MSI-H or dMMR cancers; and the treatment comprises administering to the subject pembrolizumab.

In certain embodiments, the subject is undergoing or has undergone treatment for colorectal cancer (CRC); and the treatment comprises administering to the subject one or more of ipilimumab or nivolumab.

In certain embodiments, the subject is undergoing or has undergone treatment for metastatic urothelial carcinoma (mUC); and the treatment comprises administering to the subject one or more of nivolumab, pembrolizumab, atezolizumab, avelumab, or durvalumab.

In certain embodiments, the subject is undergoing or has undergone treatment for cervical cancer; and the treatment comprises administering to the subject pembrolizumab.

In certain embodiments, the subject is undergoing or has undergone treatment for SCCHN; and the treatment comprises administering to the subject one or more of pembrolizumab or nivolumab.

In certain embodiments, the subject is undergoing or has undergone treatment for gastric or gastroesophageal junction (GEJ) adenocarcinoma; and the treatment comprises administering to the subject pembrolizumab.

In certain embodiments, the subject is undergoing or has undergone treatment for renal cell carcinoma (RCC); and the treatment comprises administering to the subject one or more of ipilimumab or nivolumab.

In certain embodiments, the subject is undergoing or has undergone treatment for classical Hodgkin Lymphoma (CHL); and the treatment comprises administering to the subject one or more of pembrolizumab or nivolumab.

In certain embodiments, the subject is undergoing or has undergone treatment for primary mediastinal large B-cell lymphoma (PMBCL); and the treatment comprises administering to the subject pembrolizumab.

In certain embodiments, the subject is undergoing or has undergone treatment for triple-negative breast cancer (TNBC); and the treatment comprises administering to the subject atezolizumab.

Pharmaceutical Compositions and Administration

General

The chemical entity, or a pharmaceutically acceptable salt and/or cocrystal thereof is administered to a subject in need thereof by any route which makes the chemical entity bioavailable (e.g., locally bioavailable). In certain embodiments, the route is oral administration. In certain embodiments, the route is rectal administration.

In some embodiments, the chemical entity, or a pharmaceutically acceptable salt and/or cocrystal thereof is administered as a pharmaceutical composition that includes the chemical entity and one or more pharmaceutically acceptable excipients, and optionally one or more other therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of chemical entities described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumor, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

In some embodiments, the chemical entity can be formulated and/or administered as described anywhere herein. In some embodiments, the chemical entity, or a pharmaceutically acceptable salt thereof, is administered by oral administration. In some embodiments, the chemical entity, or a pharmaceutically acceptable salt thereof, is administered by tablet or pill.

In some embodiments, the method herein comprises administering an effective amount of the chemical entity, or a pharmaceutically acceptable salt thereof, to the GI tract of the subject. In some embodiments, the method herein comprises locally administering an effective amount of the chemical entity, or a pharmaceutically acceptable salt thereof to the GI tract of the subject. In some embodiments, the method herein comprises topically administering an effective amount of the chemical entity, or a pharmaceutically acceptable salt thereof to the GI tract of the subject.

In some embodiments, the chemical entity, or a pharmaceutically acceptable salt thereof, is administered by rectal administration. In certain embodiments, the chemical entity, or a pharmaceutically acceptable salt thereof, is administered by enema, rectal gel, rectal foam, rectal aerosol, or suppository. In certain embodiments, the chemical entity or a pharmaceutically acceptable salt thereof, is administered by enema.

Local Administration

In some embodiments, the chemical entities described herein or a pharmaceutical composition thereof are suitable for local administration, e.g., local administration by way of administering the chemical entities or composition thereof at a particular treatment site, (e.g., the digestive tract, the gastrointestinal (“GI”) tract, e.g., colon) so as to provide local administration of the chemical entity to the area in need of treatment (e.g., oral cavity; GI tract, e.g., the colon; eye; skin; or joint). In certain embodiments, relatively low systemic exposure of the chemical entities occurs during said local administration. Examples of such compositions include, e.g., compositions for oral administration. Examples of such compositions also include e.g., compositions for rectal administration.

In some embodiments, the chemical entities described herein or a pharmaceutical composition thereof are suitable for local administration to the GI tract, e.g., colon. In certain embodiments, upon administration, the local concentration of the chemical entities in the GI tract is higher (e.g., from about 2 times higher to about 1,000 times higher; from about 2 times higher to about 900 times higher; from about 2 times higher to about 800 times higher; from about 2 times higher to about 700 times higher; from about 2 times higher to about 500 times higher; from about 2 times higher to about 400 times higher; from about 2 times higher to about 300 times higher; from about 2 times higher to about 200 times higher; from about 2 times higher to about 100 times higher; from about 2 times higher to about 50 times higher, from about 5 times higher to about 1,000 times higher; from about 5 times higher to about 900 times higher; from about 5 times higher to about 800 times higher; from about 2 times higher to about 700 times higher; from about 5 times higher to about 500 times higher; from about 5 times higher to about 400 times higher; from about 5 times higher to about 300 times higher; from about 5 times higher to about 200 times higher; from about 5 times higher to about 100 times higher; from about 5 times higher to about 50 times higher; from about 5 times higher to about 25 times higher; from about 5 times higher to about 15 times higher; e.g., about 1,000 times higher, about 900 times higher, about 800 times higher, about 700 times higher, about 600 times higher, about 500 times higher, about 400 times higher, about 300 times higher, about 200 times higher, about 100 times higher, about 50 times higher, about 25 time higher, about 20 times higher, about 15 times higher, about 10 times higher, about 5 times higher) than the concentration of the chemical entity in the plasma compartment. In certain of these embodiments, the chemical entity in the plasma compartment is subject to first pass metabolism.

In some embodiments, the chemical entities described herein or a pharmaceutical composition thereof are suitable for local administration to one or more specific locations within the digestive or GI tract, e.g., colon. For example, at least some of the chemical entity is present in the upper GI tract (e.g., stomach); or at least some of the chemical entity is present in the lower GI tract (e.g., the large intestine, e.g., the colon, e.g., the ascending colon and/or transverse colon and/or distal colon; or the small bowel). As a further example, at least some of the chemical entity is present in the ascending colon and/or the transverse colon and/or the distal colon and/or the small bowel and/or the stomach. Methods of said local administration can include, without limitation, oral administration and/or rectal administration.

In one aspect, provided herein is a composition comprising the chemical entity as described anywhere herein and one or more pharmaceutically acceptable excipients, wherein the composition is suitable for oral administration.

In one aspect, provided herein is a composition comprising the chemical entity as described anywhere herein and one or more pharmaceutically acceptable excipients, wherein the composition is suitable for local, topical administration. In certain embodiments, the chemical entities described herein or a pharmaceutical composition thereof are suitable for rectal administration. Rectal compositions include, without limitation, enemas, rectal gels, rectal foams, rectal aerosols, suppositories, jelly suppositories, and enemas (e.g., retention enemas).

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In some embodiments, administration of a single dose of the composition to a subject produces a local concentration of the chemical entity in the GI tract (e.g., colon) of the subject that is higher than the concentration of the chemical entity in the plasma compartment of the subject.

In some embodiments, administration of a single dose of the composition to a subject produces a local concentration of the chemical entity in the GI tract (e.g., colon) of the subject that is at least about 200 times higher than the concentration of the chemical entity in the plasma compartment of the subject.

In some embodiments, administration of a single dose of the composition to a subject produces a local concentration of the chemical entity in the GI tract (e.g., colon) of the subject that is at least about 300 times higher than the concentration of the chemical entity in the plasma compartment of the subject.

In some embodiments, administration of a single dose of the composition to a subject produces a local concentration of the chemical entity in the GI tract (e.g., colon) of the subject that is at least about 500 times higher than the concentration of the chemical entity in the plasma compartment of the subject.

In some embodiments, administration of a single dose of the composition to a subject produces a local concentration of the chemical entity in the GI tract (e.g., colon) of the subject that is at least about 700 times higher than the concentration of the chemical entity in the plasma compartment of the subject.

In another aspect, provided herein is a dosage form (e.g., a unit dosage form) comprising a composition as described anywhere herein, wherein the dosage form is suitable for oral administration.

In another aspect, provided herein is a dosage form (e.g., a unit dosage form) comprising a composition as described anywhere herein, wherein the dosage form is suitable for rectal administration.

In some embodiments, the dosage form further comprises one or more components that chemically and/or structurally predispose the dosage form for delivery of the chemical entity to the ascending colon.

In some embodiments, the dosage form further comprises one or more components that chemically and/or structurally predispose the dosage form for delivery of the chemical entity to the transverse colon.

In some embodiments, the dosage form further comprises one or more components that chemically and/or structurally predispose the dosage form for delivery of the chemical entity to the distal colon.

In some embodiments, the dosage form further comprises one or more components that chemically and/or structurally predispose the dosage form for delivery of the chemical entity to the small bowel.

Oral Delivery

In other embodiments, the chemical entities described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Liquid dosage forms for oral administration include pharmaceutically acceptable emulsions, microemulsions, solutions, suspensions, syrups and elixirs. In addition to the chemical entities described herein, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. In certain embodiments, the liquid dosage form is a mouthwash. In certain embodiments, such liquid oral dosage forms are useful for local and topical administration to the digestive or GI tract, e.g., digestive tract, e.g., oral cavity.

Enema Formulations

In some embodiments, enema formulations containing the chemical entities described herein are provided in “ready-to-use” form.

In some embodiments, enema formulations containing the chemical entities described herein are provided in one or more kits or packs. In certain embodiments, the kit or pack includes two or more separately contained/packaged components, e.g. two components, which when mixed together, provide the desired formulation (e.g., as a suspension). In certain of these embodiments, the two component system includes a first component and a second component, in which: (i) the first component (e.g., contained in a sachet) includes the chemical entity (as described anywhere herein) and optionally one or more pharmaceutically acceptable excipients (e.g., together formulated as a solid preparation, e.g., together formulated as a wet granulated solid preparation); and (ii) the second component (e.g., contained in a vial or bottle) includes one or more liquids and optionally one or more other pharmaceutically acceptable excipients together forming a liquid carrier. Prior to use (e.g., immediately prior to use), the contents of (i) and (ii) are combined to form the desired enema formulation, e.g., as a suspension. In other embodiments, each of component (i) and (ii) is provided in its own separate kit or pack.

In some embodiments, each of the one or more liquids is water, or a physiologically acceptable solvent, or a mixture of water and one or more physiologically acceptable solvents. Typical such solvents include, without limitation, glycerol, ethylene glycol, propylene glycol, polyethylene glycol and polypropylene glycol. In certain embodiments, each of the one or more liquids is water. In other embodiments, each of the one or more liquids is an oil, e.g. natural and/or synthetic oils that are commonly used in pharmaceutical preparations.

Further pharmaceutical excipients and carriers that may be used in the pharmaceutical products herein described are listed in various handbooks (e.g. D. E. Bugay and W. P. Findlay (Eds) Pharmaceutical excipients (Marcel Dekker, New York, 1999), E-M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe für Pharmazie, Kosmetik and angrenzende Gebiete (Edition Cantor Aulendorf, 1989)).

In some embodiments, each of the one or more pharmaceutically acceptable excipients can be independently selected from thickeners, viscosity enhancing agents, bulking agents, mucoadhesive agents, penetration enhancers, buffers, preservatives, diluents, binders, lubricants, glidants, disintegrants, fillers, solubilizing agents, pH modifying agents, preservatives, stabilizing agents, anti-oxidants, wetting or emulsifying agents, suspending agents, pigments, colorants, isotonic agents, chelating agents, emulsifiers, and diagnostic agents.

In certain embodiments, each of the one or more pharmaceutically acceptable excipients can be independently selected from thickeners, viscosity enhancing agents, mucoadhesive agents, buffers, preservatives, diluents, binders, lubricants, glidants, disintegrants, and fillers.

In certain embodiments, each of the one or more pharmaceutically acceptable excipients can be independently selected from thickeners, viscosity enhancing agents, bulking agents, mucoadhesive agents, buffers, preservatives, and fillers.

In certain embodiments, each of the one or more pharmaceutically acceptable excipients can be independently selected from diluents, binders, lubricants, glidants, and disintegrants.

Examples of thickeners, viscosity enhancing agents, and mucoadhesive agents include without limitation: gums, e.g. xanthan gum, guar gum, locust bean gum, tragacanth gums, karaya gum, ghatti gum, cholla gum, psyllium seed gum and gum arabic; poly(carboxylic acid-containing) based polymers, such as poly (acrylic, maleic, itaconic, citraconic, hydroxyethyl methacrylic or methacrylic) acid which have strong hydrogen-bonding groups, or derivatives thereof such as salts and esters; cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof; clays such as manomorillonite clays, e.g. Veegun, attapulgite clay; polysaccharides such as dextran, pectin, amylopectin, agar, mannan or polygalactonic acid or starches such as hydroxypropyl starch or carboxymethyl starch; polypeptides such as casein, gluten, gelatin, fibrin glue; chitosan, e.g. lactate or glutamate or carboxymethyl chitin; glycosaminoglycans such as hyaluronic acid; metals or water soluble salts of alginic acid such as sodium alginate or magnesium alginate; schleroglucan; adhesives containing bismuth oxide or aluminium oxide; atherocollagen; polyvinyl polymers such as carboxyvinyl polymers; polyvinylpyrrolidone (povidone); polyvinyl alcohol; polyvinyl acetates, polyvinylmethyl ethers, polyvinyl chlorides, polyvinylidenes, and/or the like; polycarboxylated vinyl polymers such as polyacrylic acid as mentioned above; polysiloxanes; polyethers; polyethylene oxides and glycols; polyalkoxys and polyacrylamides and derivatives and salts thereof. Preferred examples can include cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof (e.g., methyl cellulose); and polyvinyl polymers such as polyvinylpyrrolidone (povidone).

Examples of preservatives include without limitation: benzalkonium chloride, benzoxonium chloride, benzethonium chloride, cetrimide, sepazonium chloride, cetylpyridinium chloride, domiphen bromide (Bradosol®), thiomersal, phenylmercuric nitrate, phenylmercuric acetate, phenylmercuric borate, methylparaben, propylparaben, chlorobutanol, benzyl alcohol, phenyl ethyl alcohol, chlorohexidine, polyhexamethylene biguanide, sodium perborate, imidazolidinyl urea, sorbic acid, Purite®), Polyquart®), and sodium perborate tetrahydrate and the like.

In certain embodiments, the preservative is a paraben, or a pharmaceutically acceptable salt thereof. In some embodiments, the paraben is an alkyl substituted 4-hydroxybenzoate, or a pharmaceutically acceptable salt or ester thereof. In certain embodiments, the alkyl is a C1-C4 alkyl. In certain embodiments, the preservative is methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof.

Examples of buffers include without limitation: phosphate buffer system (sodium dihydrogen phosphate dehydrate, disodium phosphate dodecahydrate, bibasic sodium phosphate, anhydrous monobasic sodium phosphate), bicarbonate buffer system, and bisulfate buffer system.

Examples of disintegrants include, without limitation: carmellose calcium, low substituted hydroxypropyl cellulose (L-HPC), carmellose, croscarmellose sodium, partially pregelatinized starch, dry starch, carboxymethyl starch sodium, crospovidone, polysorbate 80 (polyoxyethylenesorbitan oleate), starch, sodium starch glycolate, hydroxypropyl cellulose pregelatinized starch, clays, cellulose, alginine, gums or cross linked polymers, such as cross-linked PVP (Polyplasdone XL from GAF Chemical Corp). In certain embodiments, the disintegrant is crospovidone.

Examples of glidants and lubricants (aggregation inhibitors) include without limitation: talc, magnesium stearate, calcium stearate, colloidal silica, stearic acid, aqueous silicon dioxide, synthetic magnesium silicate, fine granulated silicon oxide, starch, sodium laurylsulfate, boric acid, magnesium oxide, waxes, hydrogenated oil, polyethylene glycol, sodium benzoate, stearic acid glycerol behenate, polyethylene glycol, and mineral oil. In certain embodiments, the glidant/lubricant is magnesium stearate, talc, and/or colloidal silica; e.g., magnesium stearate and/or talc.

Examples of diluents, also referred to as “fillers” or “bulking agents” include without limitation: dicalcium phosphate dihydrate, calcium sulfate, lactose (e.g., lactose monohydrate), sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. In certain embodiments, the diluent is lactose (e.g., lactose monohydrate).

Examples of binders include without limitation: starch, pregelatinized starch, gelatin, sugars (including sucrose, glucose, dextrose, lactose and sorbitol), polyethylene glycol, waxes, natural and synthetic gums such as acacia tragacanth, sodium alginate cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone (povidone). In certain embodiments, the binder is polyvinylpyrrolidone (povidone).

In some embodiments, enema formulations containing the chemical entities described herein include water and one or more (e.g., all) of the following excipients:
One or more (e.g., one, two, or three) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);
One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof;
One or more (e.g., one or two; e.g., two) buffers, such as phosphate buffer system (e.g., sodium dihydrogen phosphate dehydrate, disodium phosphate dodecahydrate);
One or more (e.g., one or two, e.g., two) glidants and/or lubricants, such as magnesium stearate and/or talc;
One or more (e.g., one or two; e.g., one) disintegrants, such as crospovidone; and
One or more (e.g., one or two; e.g., one) diluents, such as lactose (e.g., lactose monohydrate).

In certain of these embodiments, the chemical entity is a chemical entity as described herein, or a pharmaceutically acceptable salt and/or hydrate and/or cocrystal thereof.

In certain embodiments, enema formulations containing the chemical entities described herein include water, methyl cellulose, povidone, methylparaben, propylparaben, sodium dihydrogen phosphate dehydrate, disodium phosphate dodecahydrate, crospovidone, lactose monohydrate, magnesium stearate, and talc. In certain of these embodiments, the chemical entity is a chemical entity as described herein, or a pharmaceutically acceptable salt and/or hydrate and/or cocrystal thereof.

In certain embodiments, enema formulations containing the chemical entities described herein are provided in one or more kits or packs. In certain embodiments, the kit or pack includes two separately contained/packaged components, which when mixed together, provide the desired formulation (e.g., as a suspension). In certain of these embodiments, the two component system includes a first component and a second component, in which: (i) the first component (e.g., contained in a sachet) includes the chemical entity (as described anywhere herein) and one or more pharmaceutically acceptable excipients (e.g., together formulated as a solid preparation, e.g., together formulated as a wet granulated solid preparation); and (ii) the second component (e.g., contained in a vial or bottle) includes one or more liquids and one or more one or more other pharmaceutically acceptable excipients together forming a liquid carrier. In other embodiments, each of component (i) and (ii) is provided in its own separate kit or pack.

In certain of these embodiments, component (i) includes the chemical entity, or a pharmaceutically acceptable salt and/or hydrate and/or cocrystal thereof and one or more (e.g., all) of the following excipients:
(a) One or more (e.g., one) binders (e.g., a polyvinyl polymer, such as polyvinylpyrrolidone (povidone);
(b) One or more (e.g., one or two, e.g., two) glidants and/or lubricants, such as magnesium stearate and/or talc;
(c) One or more (e.g., one or two; e.g., one) disintegrants, such as crospovidone; and
(d) One or more (e.g., one or two; e.g., one) diluents, such as lactose (e.g., lactose monohydrate).

In certain embodiments, component (i) includes from about 40 weight percent to about 80 weight percent (e.g., from about 50 weight percent to about 70 weight percent, from about 55 weight percent to about 70 weight percent; from about 60 weight percent to about 65 weight percent; e.g., about 62.1 weight percent) of the chemical entity, or a pharmaceutically acceptable salt and/or hydrate and/or cocrystal thereof).

In certain embodiments, component (i) includes from about 0.5 weight percent to about 5 weight percent (e.g., from about 1.5 weight percent to about 4.5 weight percent, from about 2 weight percent to about 3.5 weight percent; e.g., about 2.76 weight percent) of the binder (e.g., povidone).

In certain embodiments, component (i) includes from about 0.5 weight percent to about 5 weight percent (e.g., from about 0.5 weight percent to about 3 weight percent, from about 1 weight percent to about 3 weight percent; about 2 weight percent e.g., about 1.9 weight percent) of the disintegrant (e.g., crospovidone).

In certain embodiments, component (i) includes from about 10 weight percent to about 50 weight percent (e.g., from about 20 weight percent to about 40 weight percent, from about 25 weight percent to about 35 weight percent; e.g., about 31.03 weight percent) of the diluent (e.g., lactose, e.g., lactose monohydrate).

In certain embodiments, component (i) includes from about 0.05 weight percent to about 5 weight percent (e.g., from about 0.05 weight percent to about 3 weight percent) of the glidants and/or lubricants.

In certain embodiments (e.g., when component (i) includes one or more lubricants, such as magnesium stearate), component (i) includes from about 0.05 weight percent to about 1 weight percent (e.g., from about 0.05 weight percent to about 1 weight percent; from about 0.1 weight percent to about 1 weight percent; from about 0.1 weight percent to about 0.5 weight percent; e.g., about 0.27 weight percent) of the lubricant (e.g., magnesium stearate).

In certain embodiments (when component (i) includes one or more lubricants, such as talc), component (i) includes from about 0.5 weight percent to about 5 weight percent (e.g., from about 0.5 weight percent to about 3 weight percent, from about 1 weight percent to about 3 weight percent; from about 1.5 weight percent to about 2.5 weight percent; from about 1.8 weight percent to about 2.2 weight percent; about 1.93 weight percent) of the lubricant (e.g., talc).

In certain of these embodiments, each of (a), (b), (c), and (d) above is present.

In certain embodiments, component (i) includes the ingredients and amounts as shown in Table 7.

TABLE 7

| Ingredient | Weight Percent |
|---|---|
| Chemical ntity | 40 weight percent to about 80 weight |
| | percent (e.g., from about 50 weight percent |
| | to about 70 weight percent, from about 55 |
| | weight percent to about 70 weight percent; |
| | from about 60 weight percent to about 65 |
| | weight percent; e.g., about 62.1 weight |
| | percent) |
| Crospovidone (Kollidon CL) | 0.5 weight percent to about 5 weight |
| | percent (e.g., from about 0.5 weight |
| | percent to about 3 weight percent, from |
| | about 1 weight percent to about 3 weight |
| | percent; about 1.93 weight percent |
| lactose monohydrate | about 10 weight percent to about 50 weight |
| (Pharmatose 200M) | percent (e.g., from about 20 weight percent |
| | to about 40 weight percent, from about 25 |
| | weight percent to about 35 weight percent; |
| | e.g., about 31.03 weight percent |
| Povidone (Kollidon K30) | about 0.5 weight percent to about 5 weight |
| | percent (e.g., from about 1.5 weight |
| | percent to about 4.5 weight percent, from |
| | about 2 weight percent to about 3.5 weight |
| | percent; e.g., about 2.76 weight percent |
| talc | 0.5 weight percent to about 5 weight |
| | percent (e.g., from about 0.5 weight |
| | percent to about 3 weight percent, from |
| | about 1 weight percent to about 3 weight |
| | percent; from about 1.5 weight percent to |
| | about 2.5 weight percent; from about 1.8 |
| | weight percent to about 2.2 weight |
| | percent;e.g., about 1.93 weight percent |
| Magnesium stearate | about 0.05 weight percent to about 1 |
| | weight percent (e.g., from about 0.05 |
| | weight percent to about 1 weight percent; |
| | from about 0.1 weight percent to about 1 |
| | weight percent; from about 0.1 weight |
| | percent to about 0.5 weight percent; e.g., |
| | about 0.27 weight percent |

In certain embodiments, component (i) includes the ingredients and amounts as shown in Table 8.

TABLE 8

| Ingredient | Weight Percent |
|---|---|
| Chemical entity | About 62.1 weight percent) |
| Crospovidone (Kollidon CL) | About 1.93 weight percent |
| lactose monohydrate | About 31.03 weight percent |
| (Pharmatose 200M) | |
| Povidone (Kollidon K30) | About 2.76 weight percent |
| talc | About 1.93 weight percent |
| Magnesium stearate | About 0.27 weight percent |

In certain embodiments, component (i) is formulated as a wet granulated solid preparation. In certain of these embodiments an internal phase of ingredients (the chemical entity, disintegrant, and diluent) are combined and mixed in a high-shear granulator. A binder (e.g., povidone) is dissolved in water to form a granulating solution. This solution is added to the Inner Phase mixture resulting in the development of granules. While not wishing to be bound by theory, granule development is believed to be facilitated by the interaction of the polymeric binder with the materials of the internal phase. Once the granulation is formed and dried, an external phase (e.g., one or more lubricants—not an intrinsic component of the dried granulation), is added to the dry granulation. It is believed that lubrication of the granulation is important to the flowability of the granulation, in particular for packaging. See, e.g., Example 8.

In certain of the foregoing embodiments, component (ii) includes water and one or more (e.g., all) of the following excipients:
(a′) One or more (e.g., one, two; e.g., two) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);
(b′) One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof; and
(c′) One or more (e.g., one or two; e.g., two) buffers, such as phosphate buffer system (e.g., sodium dihydrogen phosphate dihydrate, disodium phosphate dodecahydrate);

In certain of the foregoing embodiments, component (ii) includes water and one or more (e.g., all) of the following excipients:
(a″) a first thickener, viscosity enhancing agent, binder, and/or mucoadhesive agent (e.g., a cellulose or cellulose ester or ether or derivative or salt thereof (e.g., methyl cellulose));
(a′″) a second thickener, viscosity enhancing agent, binder, and/or mucoadhesive agent (e.g., a polyvinyl polymer, such as polyvinylpyrrolidone (povidone));
(b″) a first preservative, such as a paraben, e.g., propyl 4-hydroxybenzoate (propylparaben), or a pharmaceutically acceptable salt or ester thereof;
(b″) a second preservative, such as a paraben, e.g., methyl 4-hydroxybenzoate (methylparaben), or a pharmaceutically acceptable salt or ester thereof,
(c″) a first buffer, such as phosphate buffer system (e.g., disodium phosphate dodecahydrate);
(c′″) a second buffer, such as phosphate buffer system (e.g., sodium dihydrogen phosphate dehydrate),

In certain embodiments, component (ii) includes from about 0.05 weight percent to about 5 weight percent (e.g., from about 0.05 weight percent to about 3 weight percent, from about 0.1 weight percent to about 3 weight percent; e.g., about 1.4 weight percent) of (a″).

In certain embodiments, component (ii) includes from about 0.05 weight percent to about 5 weight percent (e.g., from about 0.05 weight percent to about 3 weight percent, from about 0.1 weight percent to about 2 weight percent; e.g., about 1.0 weight percent) of (a′″).

In certain embodiments, component (ii) includes from about 0.005 weight percent to about 0.1 weight percent (e.g., from about 0.005 weight percent to about 0.05 weight percent; e.g., about 0.02 weight percent) of (b″).

In certain embodiments, component (ii) includes from about 0.05 weight percent to about 1 weight percent (e.g., from about 0.05 weight percent to about 0.5 weight percent; e.g., about 0.20 weight percent) of (b′″).

In certain embodiments, component (ii) includes from about 0.05 weight percent to about 1 weight percent (e.g., from about 0.05 weight percent to about 0.5 weight percent; e.g., about 0.15 weight percent) of (c″).

In certain embodiments, component (ii) includes from about 0.005 weight percent to about 0.5 weight percent (e.g., from about 0.005 weight percent to about 0.3 weight percent; e.g., about 0.15 weight percent) of (c′″).

In certain of these embodiments, each of (a″)-(c′″) is present.

In certain embodiments, component (ii) includes water (up to 100%) and the ingredients and amounts as shown in Table 9.

TABLE 9

| Ingredient | Weight Percent |
|---|---|
| methyl cellulose | 0.05 weight percent to about 5 weight |
| (Methocel A15C premium) | percent (e.g., from about 0.05 weight |
| | percent to about 3 weight percent, from |
| | about 0.1 weight percent to about 3 weight |
| | percent; e.g., about 1.4 weight percent |
| Povidone (Kollidon K30) | 0.05 weight percent to about 5 weight |
| | percent (e.g., from about 0.05 weight |
| | percent to about 3 weight percent, from |
| | about 0.1 weight percent to about 2 weight |
| | percent; e.g., about 1.0 weight percent |
| propyl 4-hydroxybenzoate | about 0.005 weight percent to about 0.1 |
| | weight percent (e.g., from about 0.005 |
| | weight percent to about 0.05 weight |
| | percent; e.g., about 0.02 weight percent) |
| methyl 4-hydroxybenzoate | about 0.05 weight percent to about 1 |
| | weight percent (e.g., from about 0.05 |
| | weight percent to about 0.5 weight percent; |
| | e.g., about 0.20 weight percent) |
| disodium phosphate | about 0.05 weight percent to about 1 |
| dodecahydrate | weight percent (e.g., from about 0.05 |
| | weight percent to about 0.5 weight percent; |
| | e.g., about 0.15 weight percent) |
| sodium dihydrogen | about 0.005 weight percent to about 0.5 |
| phospahate dihydrate | weight percent (e.g., from about 0.005 |
| | weight percent to about 0.3 weight percent; |
| | e.g., about 0.15 weight percent) |

In certain embodiments, component (ii) includes water (up to 100%) and the ingredients and amounts as shown in Table 10.

TABLE 10

| Ingredient | Weight Percent |
|---|---|
| methyl cellulose (Methocel A15C | about 1.4 weight percent |
| premium) | |
| Povidone (Kollidon K30) | about 1.0 weight percent |
| propyl 4-hydroxybenzoate | about 0.02 weight percent |
| methyl 4-hydroxybenzoate | about 0.20 weight percent |
| disodium phosphate dodecahydrate | about 0.15 weight percent |
| sodium dihydrogen phospahate dihydrate | about 0.15 weight percent |

Ready-to-use” enemas are generally be provided in a “single-use” sealed disposable container of plastic or glass. Those formed of a polymeric material preferably have sufficient flexibility for ease of use by an unassisted patient. Typical plastic containers can be made of polyethylene. These containers may comprise a tip for direct introduction into the rectum. Such containers may also comprise a tube between the container and the tip. The tip is preferably provided with a protective shield which is removed before use. Optionally the tip has a lubricant to improve patient compliance.

In some embodiments, the enema formulation (e.g., suspension) is poured into a bottle for delivery after it has been prepared in a separate container. In certain embodiments, the bottle is a plastic bottle (e.g., flexible to allow for delivery by squeezing the bottle), which can be a polyethylene bottle (e.g., white in color). In some embodiments, the bottle is a single chamber bottle, which contains the suspension or solution. In other embodiments, the bottle is a multichamber bottle, where each chamber contains a separate mixture or solution. In still other embodiments, the bottle can further include a tip or rectal cannula for direct introduction into the rectum. In some embodiments, the enema formulation can be delivered in the device shown in FIGS. 1A-1C, which includes a plastic bottle, a breakable capsule, and a rectal cannula and single flow pack.

Other Forms of Delivery

In some embodiments, the chemical entities described herein or a pharmaceutical composition thereof are suitable for local and topical administration to the eye (e.g., eye drops). Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

In some embodiments, the chemical entities described herein or a pharmaceutical composition thereof are suitable for local and topical administration to skin (e.g., ointments and creams). Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

Dosages

The dosages may be varied depending on the requirement of the patient, the severity of the condition being treating and the particular chemical entity being employed. Determination of the proper dosage for a particular situation can be determined by one skilled in the medical arts. The total daily dosage may be divided and administered in portions throughout the day or by means providing continuous delivery.

In some embodiments, the chemical entity is administered is administered at a dosage of from about 0.01 mg/Kg to about 200 mg/Kg (e.g., from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg).

In certain embodiments, the chemical entity is administered at a dosage of from about 15 mg/Kg to about 100 mg/Kg (e.g., from about 15 mg/Kg to about 90 mg/Kg, from about 20 mg/Kg to about 100 mg/Kg; from about 20 mg/Kg to about 90 mg/Kg; from about 20 mg/Kg to about 80 mg/Kg; from about 30 mg/Kg to about 90 mg/Kg; from about 30 mg/Kg to about 80 mg/Kg; from about 35 mg/Kg to about 75 mg/Kg; from about 10 mg/Kg to about 50 mg/Kg; from about 15 mg/Kg to about 45 mg/Kg; e.g., about 35 mg/Kg or about 75 mg/Kg). In other embodiments, the chemical entity is administered at a dosage of from about 0.1 mg/Kg to about 10 mg/Kg (e.g., from about 0.1 mg/Kg to about 5 mg/Kg; from about 1 mg/Kg to about 10 mg/Kg; from about 1 mg/Kg to about 5 mg/Kg).

In some embodiments, formulations include from about 0.5 mg to about 2500 mg (e.g., from about 0.5 mg to about 2000 mg, from about 0.5 mg to about 1000 mg, from about 0.5 mg to about 750 mg, from about 0.5 mg to about 600 mg, from about 0.5 mg to about 500 mg, from about 0.5 mg to about 400 mg, from about 0.5 mg to about 300 mg, from about 0.5 mg to about 200 mg; e.g., from about 5 mg to about 2500 mg, from about 5 mg to about 2000 mg, from about 5 mg to about 1000 mg; from about 5 mg to about 750 mg; from about 5 mg to about 600 mg; from about 5 mg to about 500 mg; from about 5 mg to about 400 mg; from about 5 mg to about 300 mg; from about 5 mg to about 200 mg; e.g., from about 50 mg to about 2000 mg, from about 50 mg to about 1000 mg, from about 50 mg to about 750 mg, from about 50 mg to about 600 mg, from about 50 mg to about 500 mg, from about 50 mg to about 400 mg, from about 50 mg to about 300 mg, from about 50 mg to about 200 mg; e.g., from about 100 mg to about 2500 mg, from about 100 mg to about 2000 mg, from about 100 mg to about 1000 mg, from about 100 mg to about 750 mg, from about 100 mg to about 700 mg, from about 100 mg to about 600 mg, from about 100 mg to about 500 mg, from about 100 mg to about 400 mg, from about 100 mg to about 300 mg, from about 100 mg to about 200 mg; e.g., from about 150 mg to about 2500 mg, from about 150 mg to about 2000 mg, from about 150 mg to about 1000 mg, from about 150 mg to about 750 mg, from about 150 mg to about 700 mg, from about 150 mg to about 600 mg, from about 150 mg to about 500 mg, from about 150 mg to about 400 mg, from about 150 mg to about 300 mg, from about 150 mg to about 200 mg; e.g., from about 150 mg to about 500 mg; e.g., from about 300 mg to about 2500 mg, from about 300 mg to about 2000 mg, from about 300 mg to about 1000 mg, from about 300 mg to about 750 mg, from about 300 mg to about 700 mg, from about 300 mg to about 600 mg; e.g., from about 400 mg to about 2500 mg, from about 400 mg to about 2000 mg, from about 400 mg to about 1000 mg, from about 400 mg to about 750 mg, from about 400 mg to about 700 mg, from about 400 mg to about 600 from about 400 mg to about 500 mg; e.g., 150 mg or 450 mg) of the chemical entity.

In certain embodiments, formulations include from about 50 mg to about 250 mg (e.g., from about 100 mg to about 200; e.g., about 150 mg) of the chemical entity.

In certain embodiments, enema formulations include from about 350 mg to about 550 mg (e.g., from about 400 mg to about 500; e.g., about 450 mg) of the chemical entity.

The foregoing dosages can be administered on a daily basis (e.g., as a single dose per day; or as two or more divided doses per day; or a two or more doses; e.g., two doses per day) or non-daily basis (e.g., every other day, every two days, every three days, once weekly, twice weeks, once every two weeks, once a month). In certain embodiments, dosages can be administered for about 1 week, about 2 weeks, about 3 weeks, about 4 weeks, about 5 weeks, about 6 weeks, about 7 weeks, about 8 weeks, about 3 months, about 6 months, about 1 year, or beyond. For example, dosages (e.g., about 2.5 mg/mL or about 7.5 mg/mL) of the chemical entity in liquid carrier can be administered twice a day on a daily basis for about 6 weeks. For example, about 2.5 mg/mL or about 7.5 mg/mL of the chemical entity in liquid carrier can be administered twice a day on a daily basis for about 6 weeks. Representative liquid carriers include, e.g., those previously described in conjunction with component (ii).

### Combination Therapy

In some embodiments, the methods described herein can further include administering a second therapeutic agent or regimen.

In certain embodiments, the second therapeutic agent or regimen is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the chemical entity. By way of example, the second therapeutic agent or regimen and the chemical entity are provided to the subject simultaneously in the same dosage form. As another example, the second therapeutic agent or regimen and the chemical entity are provided to the subject concurrently in separate dosage forms.

In still other embodiments, the second therapeutic agent or regimen is administered to the subject after contacting with or administering the chemical entity (e.g., about one hour after, or about 6 hours after, or about 12 hours after, or about 24 hours after, or about 48 hours after, or about 1 week after, or about 1 month after).

In certain embodiments, the second therapeutic agent is a chemotherapeutic immunomodulator, e.g., an immune checkpoint inhibitor, which can be as defined anywhere herein. In other embodiments, the second therapeutic agent or regimen is one or more anti-inflammatory agents or immunomodulator acting locally in the GI tract. In other embodiments, the second therapeutic agent or regimen is 5-ASA (and associated delivery systems), anti-SMAD7 antisense, orally formulated anti-TNFs, anti-integrins, sulfasalazine, balsalazide, steroids, azathioprine, and methotrexate. In further embodiments, the second therapeutic agent or regimen is radiation or surgery.

In certain embodiments, the second therapeutic agent is platinum, cisplatin, carboplatin, oxaliplatin, mechlorethamine, cyclophosphamide, chlorambucil, azathioprine, mercaptopurine, vincristine, vinblastine, vinorelbine, vindesine, etoposide and teniposide, paclitaxel, docetaxel, irinotecan, topotecan, amsacrine, etoposide, etoposide phosphate, teniposide, 5-fluorouracil, leucovorin, methotrexate, gemcitabine, taxane, leucovorin, mitomycin C, tegafur-uracil, idarubicin, fludarabine, mitoxantrone, ifosfamide and doxorubicin. Additional agents include inhibitors of mTOR (mammalian target of rapamycin), including but not limited to rapamycin, everolimus, temsirolimus and deforolimus.

In some embodiments, the methods and compositions described herein are suitable for use in combination therapy with various other therapeutic regimens (e.g., chemotherapy and/or radiation). In certain embodiments, the chemical entities and methods described herein can be used to treat side effects produced by such therapeutic regimens, e.g., inflammatory bowel diseases induced by chemotherapeutic immunomodulators, e.g., checkpoint inhibitors, which in some cases can be prohibitively severe.

### Other Provisions

In some embodiments, the methods described herein are performed in the absence of a mitochondrial agent. In certain embodiments, the mitochondrial agent is a mitochondrial uncoupling agent. As a non-limiting example, the method is performed in the absence of niclosamide.

In some of embodiments, the methods described herein are performed in the absence of a compound having any one of formulas (I), (XVIII)-(XXV), and XXVII, (e.g., formula XXIV or XXV) as described in U.S. Pat. No. 10,292,951 which is incorporated herein by reference in its entirety. In certain of these embodiments, the methods described herein are performed in the absence of a niclosamide analogue having any one of formulas (I), (XVIII)-(XXV), and XXVII (e.g., formula XXIV or XXV; or XXVI) as described in U.S. Pat. No. 10,292,951 which is incorporated herein by reference in its entirety.

A number of embodiments of the invention have been described. Nevertheless, it will be understood that various modifications may be made without departing from the spirit and scope of the invention. Accordingly, other embodiments are within the scope of the following claims.

What is claimed is:

1. A method for treating iatrogenic autoimmune colitis in a subject in need thereof, the method comprising administering to the subject an effective amount of a chemical entity selected from the group consisting of:
sphingosine 1-phosphate receptor (S1P) modulators;
Janus kinase (JAK) inhibitors;
lanthionine synthetase C-like 2 (LANCL2) modulators;
integrin modulators;
immunosuppressant;
steroidal anti-inflammatory agents;
non-steroidal anti-inflammatory agents;
receptor-interacting protein kinase 1 (RIPK1) inhibitors;
EP4 modulators;
toll-like receptor modulators;
phosphatidylcholine;
Smad7 modulators;
phosphodiesterase 4 (PDE4) modulators;
tumor progression locus 2 (TPL2) inhibitors;
tyrosine kinase 2 (TYK2) inhibitors;
TEC kinase inhibitors; and
TIP60 inhibitors;
or a pharmaceutically acceptable salt thereof.

2. The method of claim 1, wherein the iatrogenic autoimmune colitis is induced by one or more chemotherapeutic agents.

3. The method of claim 2, wherein at least one of the one or more chemotherapeutic agents is a chemotherapeutic immunomodulator.

4. The method of claim 3, wherein the chemotherapeutic immunomodulator is an immune checkpoint inhibitor.

5. The method of claim 4, wherein the immune checkpoint inhibitor targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155.

6. The method of claim 4, wherein the immune checkpoint inhibitor is selected from the group consisting of: Urelumab, PF-05082566, MEDI6469, TRX518, Varlilumab, CP-870893, Pembrolizumab (PD1), Nivolumab (PD1), Atezolizumab (formerly MPDL3280A) (PDL1), MEDI4736 (PD-L1), Avelumab (PD-L1), PDR001 (PD1), BMS-986016, MGA271, Lirilumab, IPH2201, Emactuzumab, INCB024360, Galunisertib, Ulocuplumab, BKT140, Bavituximab, CC-90002, Bevacizumab, and MNRP1685A, and MGA271.

7. The method of claim 4, wherein the immune checkpoint inhibitor targets CTLA-4.

8. The method of claim 7, wherein the immune checkpoint inhibitor is an antibody.

9. The method of claim 8, wherein the antibody is ipilimumab or tremelimumab.

10. The method of claim 4, wherein the immune checkpoint inhibitor targets PD1 or PD-L1.

11. The method of claim 10, wherein the immune checkpoint inhibitor is selected from nivolumab, lambroizumab, and BMS-936559.

12. The method of any one of claims 1-11, wherein the chemical entity, or a pharmaceutically acceptable salt thereof, is a gut-restricted chemical entity.

13. The method of any one of claims 1-12, wherein the chemical entity, or a pharmaceutically acceptable salt thereof, is an intestinal-restricted chemical entity.

14. The method of any one of claims 1-13, wherein the chemical entity is a colon-restricted chemical entity.

15. The method of any one of claims 1-14, wherein the chemical entity, or a pharmaceutically acceptable salt thereof, is administered by oral administration.

16. The method of any one of claims 1-15, wherein the chemical entity, or a pharmaceutically acceptable salt thereof, is administered by tablet or pill.

17. The method of any one of claims 1-14, the method comprising administering an effective amount of the chemical entity, or a pharmaceutically acceptable salt thereof, to the GI tract of the subject.

18. The method of any one of claims 1-14 and 17, the method comprising, wherein the method comprises locally administering an effective amount of the chemical entity, or a pharmaceutically acceptable salt thereof to the GI tract of the subject.

19. The method of any one of claims 1-14, 17, and 18, wherein the method comprises topically administering an effective amount of the chemical entity, or a pharmaceutically acceptable salt thereof to the GI tract of the subject.

20. The method of any one of claims 1-14 and 17-19, wherein the chemical entity, or a pharmaceutically acceptable salt thereof, is administered by rectal administration.

21. The method of any one of claims 1-14 and 17-20, wherein the chemical entity, or a pharmaceutically acceptable salt thereof, is administered by enema, rectal gel, rectal foam, rectal aerosol, or suppository.

22. The method of any one of claims 1-14 and 17-21, wherein the chemical entity or a pharmaceutically acceptable salt thereof, is administered by enema.

23. The method of any one of claims 1-22, wherein the chemical entity or a pharmaceutically acceptable salt thereof is a sphingosine 1-phosphate receptor (S1P) modulator or a pharmaceutically acceptable salt thereof.

24. The method of claim 23, wherein the chemical entity or a pharmaceutically acceptable salt thereof is a sphingosine 1-phosphate receptor (S1P) agonist or a pharmaceutically acceptable salt thereof.

25. The method of any one of claims 23-24, wherein the chemical entity, or a pharmaceutically acceptable salt thereof is selected from the group consisting of: fingolimod; CS-0777; KKSM-07003 (KKSM-07005, KKSM-07016, SKY-59); AKP-11; CBP-307; BMS-986104; SYL-933 (SYL-933-P); cenerimod (e.g., ACT-334441); NIBR-785; BMS-520 (BMS-54) (Hou et al., Org. Process Res. Dev. 20(5): 989-995, 2016); GSK-2018682 (2018682, PPI-4621, PPI-4667, PPI-4667-P, PPI-4939, PPI-4955, or PPI-5955-P); GSK1842799 (PPI-4691); KRP-107; AMG-247 (also called AMG-277, AMG-369, and PRX-13038); ponesimod (ACT-128800, Actelion-2, R-3477, RG-3477); YP-005; mocravimod dihydrochloride (also called KNF-299, KRP-203, KRP-203-P prodrug, and mocravimod); SAR-247799; SEW2871; KRP203; siponimod (BAF-312); ozanimod (RPC 1063); ceralifimod (ONO-4641); ASP4058; GSK2018682; PF-462991 (also called PF-04629991 and PF-991); LAS-189913; LC-510201; LC-51-SPA; LC-510201; A-971432; ABT-363; OBT-893 (SH-BC-893); RP-1859 (RP-1865); ASP4085; BMS-986166; VPC-01091; CP-1050; amitriptyline; LX-2932; LX-2931 (LX-3305); KDS-1059; KSI-6666; ozanimod metabolite (e.g., RP-101074, RP-101442, RP-101988, RPC-101075, and RPC-1063); TASP-0251078 (TASP-0277308); TY-52156; amiselimod (e.g., MT-1303); NOX-S91 (NOX-S92, NOX-S93); EXEL-4541 (XL-541); VPC23019; etrasimod (e.g., APD-334 or APD-334 L-Arginine); NIBR-0213; SPG-104; BML-258; PF-543; NV-06 (idronoxil, phenoxidiol); SKI-349; B-5354a; B-5354b; B-5354c; F-12509A; VPC-94075; SCL-5081308 (SRX-224014); ABC-294640 (ABC-294735, ABC-747080, SKI-I, SKI-II, SKI-V, Yeliva®, or opaganib); SLR080811; AB-22; ONO-1266; oxfenmino hydrochloric acid; and ABT-413, or a pharmaceutically acceptable salt thereof.

26. The method of any one of claims 23-24, wherein the chemical entity or a pharmaceutically acceptable salt thereof is etrasimod.

27. The method of any one of claims 23-24, wherein the chemical entity or a pharmaceutically acceptable salt thereof is ozanimod.

28. The method of any one of claims 1-22, wherein the chemical entity or a pharmaceutically acceptable salt thereof is a Janus kinase (JAK) inhibitor.

29. The method of claim 28, wherein the chemical entity or a pharmaceutically acceptable salt thereof is selected from the group consisting of: abrocitinib, baricitinib, BMS-986165, decemotinib (VX509); filgotinib; itacitinib; oclacitinib; peficitinib; PF-06651600; PF-06700841; R333 (R932333); R348 (R932348); ruxolitinib; solcitinib; TD-1473; TD-8236; TD-5202; TD-3504; tofacitinib (e.g., tofacitinib citrate); and upadacitinib.

30. The method of any one of claims 28-29, wherein the chemical entity or a pharmaceutically acceptable salt thereof is selected from the group consisting of: TD-1473, tofacitinib, upadacitinib, filgotinib, PF-06651600, and PF-06700841.

31. The method of claim 28, wherein the chemical entity or a pharmaceutically acceptable salt thereof is a TD-1473.

32. The method of any one of claims 1-22, wherein the chemical entity or a pharmaceutically acceptable salt thereof is a lanthionine synthetase C-like 2 (LANCL2) modulator.

33. The method of claim 32, wherein the chemical entity or a pharmaceutically acceptable salt thereof is a lanthionine synthetase C-like 2 (LANCL2) agonist.

34. The method of any one of claims 32-33, wherein the chemical entity or a pharmaceutically acceptable salt thereof is selected from the group consisting of: BT-11, NX-13, and BT-13.

35. The method of any one of claims 32-34, wherein the chemical entity or a pharmaceutically acceptable salt thereof is BT-11.

36. The method of any one of claims 1-22, wherein the chemical entity or a pharmaceutically acceptable salt thereof is an integrin modulator.

37. The method of claim 36, wherein the chemical entity or a pharmaceutically acceptable salt thereof is an α4 integrin modulator or an α4β7 integrin modulator.

38. The method of claim 36, wherein the chemical entity or a pharmaceutically acceptable salt thereof is vedolizumab; natalizumab; etrolizumab; vatelizumab; PF-00547659; AJM-300; HCA2969 (carotegrast); firategrast; valategrast; R00270608; CDP-323; CT7758; GW-559090; ELND-004; PTG-100; and PN-10943.

39. The method of any one of claims 36-38, wherein the chemical entity or a pharmaceutically acceptable salt thereof is vedolizumab.

40. The method of any one of claims 1-22, wherein the chemical entity or a pharmaceutically acceptable salt thereof is an immunosuppressant.

41. The method of claim 40, wherein the chemical entity or a pharmaceutically acceptable salt thereof is selected from the group consisting of: methotrexate, azathioprine (Imuran®), cyclosporine, tacrolimus, mycophenolate mofetil (Cellcept®), and cyclophosphamide (Cytoxan®), systemic or oral corticosteroids, rapamycin, FK-506, and interferon-gamma.

42. The method of any one of claims 40-41, wherein the chemical entity or a pharmaceutically acceptable salt thereof is cyclosporine.

43. The method of any one of claims 1-22, wherein the chemical entity or a pharmaceutically acceptable salt thereof is a steroidal anti-inflammatory agent (e.g, beclomethasone 17, budesonide prednisone, prednisolone, or beclometasone dipropionate.

44. The method of any one of claims 1-22, wherein the chemical entity or a pharmaceutically acceptable salt thereof is a non-steroidal anti-inflammatory agent (e.g., 5-ASA).

45. The method of any one of claims 1-22, wherein the chemical entity or a pharmaceutically acceptable salt thereof is a receptor-interacting protein kinase 1 (RIPK1) inhibitor (e.g., GSK2982772).

46. The method of any one of claims 1-22, wherein the chemical entity or a pharmaceutically acceptable salt thereof is an EP4 modulator (e.g., KAG-308).

47. The method of any one of claims 1-22, wherein the chemical entity or a pharmaceutically acceptable salt thereof is a toll-like receptor (e.g., TLR4, TLR9) modulator (e.g., JKB-122, cobitolimod).

48. The method of any one of claims 1-22, wherein the chemical entity or a pharmaceutically acceptable salt thereof is a phosphatidylcholine (e.g., LT-02.

49. The method of any one of claims 1-22, wherein the chemical entity or a pharmaceutically acceptable salt thereof is an Smad7 modulator (e.g., mongersen).

50. The method of any one of claims 1-22, wherein the chemical entity or a pharmaceutically acceptable salt thereof is a phosphodiesterase 4 (PDE4) modulator (e.g., apremilast).

51. The method of any one of claims 1-22, wherein the chemical entity or a pharmaceutically acceptable salt thereof is a tumor progression locus 2 (TPL2) inhibitor (e.g., GS-4875).

52. The method of any one of claims 1-22, wherein the chemical entity or a pharmaceutically acceptable salt thereof is a tyrosine kinase 2 (TYK2) inhibitor (e.g., BMS-986165, PF-06700841, and PF-06826647) or a TEC kinase inhibitor (e.g., PF-06651600).

53. The method of any one of claims 1-22, wherein the chemical entity or a pharmaceutically acceptable salt thereof is a TIP60 inhibitor (see, e.g., U.S. Patent Application Publication No. 2012/0202848).

54. The method of any one of claims 1-53, wherein the method is performed in the absence of a mitochondrial agent.

55. The method of any one of claims 1-54, wherein the method is performed in the absence of niclosamide.

56. The method of any one of claims 1-55, wherein the subject is a human.

57. The method of any one of claims 1-56, wherein the subject is undergoing or has undergone treatment for cancer.

58. The method of claim 57, wherein the cancer is selected from the group consisting of: multiple myeloma, leukemias (HTLV-1 dependent, erythroleukemia, acute myelogenous leukemia (AML), chronic myelogenous leukemia (CML), and large granular lymphocyte leukemia (LGL), lymphomas (EBV-related/Burkitt's, mycosis fungoides, cutaneous T-cell lymphoma, non-Hodgkins lymphoma (NHL), anaplastic large-cell lymphoma (ALCL), breast cancers, triple-negative breast cancers, head and neck cancers, melanoma, ovarian cancers, lung cancers, pancreatic cancers, prostate cancers, sarcomas, osteosarcoma, Kaposi's sarcoma, Ewing's sarcoma, hepatocellular cancers, glioma, neuroblastoma, astrocytoma, colorectal cancers, Wilm's tumors, renal cancers, bladder cancers, endometrial cancers, cervical cancers, esophageal cancers, cutaneous squamous cell cancers, basal cell cancers, and metastatic cancers.

59. The method of claim 57, wherein the cancer is selected from the group consisting of: skin cancers (e.g., malignant melanoma (MM), Merkel cell carcinoma (MCC), cutaneous squamous cell carcinoma (CSCC)); lung cancers (e.g., non-small cell lung cancer (NSCLC) or small cell lung cancer (SCLC)); liver cancers (e.g., hepatocellular carcinoma (HCC)); MSI-H or dMMR cancers (e.g., any MSI-H or dMMR cancers, or colorectal cancer (CRC)); genitourinary cancers (e.g., metastatic urothelial carcinoma (mUC) or cervical cancer); head and neck cancers (head and neck squamous cell carcinoma (SCCHN)); gastric cancers (e.g., gastric or gastroesophageal junction (GEJ) adenocarcinoma); kidney cancers (e.g., renal cell carcinoma (RCC)); hematological cancers (e.g., classical Hodgkin Lymphoma (CHL) or primary mediastinal large B-cell lymphoma (PMBCL)); and breast cancers (e.g., triple-negative breast cancer (TNBC)).

60. The method of claim 59, wherein the cancer is selected from the group consisting of: malignant melanoma (MM), Merkel cell carcinoma (MCC), cutaneous squamous cell carcinoma (CSCC), non-small cell lung cancer (NSCLC), small cell lung cancer (SCLC), hepatocellular carcinoma (HCC), MSI-H or dMMR cancers, colorectal cancer (CRC), metastatic urothelial carcinoma (mUC) or cervical cancer, head and neck squamous cell carcinoma (SCCHN), gastric or gastroesophageal junction (GEJ) adenocarcinoma, renal cell carcinoma (RCC), classical Hodgkin Lymphoma (CHL), primary mediastinal large B-cell lymphoma (PMBCL); and triple-negative breast cancer (TNBC).

61. The method of any one of claims 57-60, wherein the treatment for cancer comprises administering to the subject an immune checkpoint inhibitor.

62. The method of claim 61, wherein the immune checkpoint inhibitor targets CTLA-4.

63. The method of claim 62, wherein the immune checkpoint inhibitor is ipilimumab or tremelimumab.

64. The method of claim 61, wherein the immune checkpoint inhibitor targets PD1 or PD-L1.

65. The method of claim 64, wherein the immune checkpoint inhibitor is selected from the group consisting of: pembrolizumab, nivolumab, atezolizumab, avelumab, lambroizumab, cemiplimab, and BMS-936559.

66. The method of claim 61, wherein the immune checkpoint inhibitor targets CD274 (e.g., the immune checkpoint inhibitor is durvalumab).

67. The method of any one of claims 1-57, wherein the subject is undergoing or has undergone treatment for malignant melanoma (e.g., metastatic melanoma); and the treatment comprises administering to the subject one or more of ipilimumab, nivolumab, or pembrolizumab.

68. The method of any one of claims 1-57, wherein the subject is undergoing or has undergone treatment for Merkel call carcinoma; and the treatment comprises administering to the subject one or more of avelumab or pembrolizumab.

69. The method of any one of claims 1-57, wherein the subject is undergoing or has undergone treatment for cutaneous squamous cell carcinoma (CSCC); and the treatment comprises administering to the subject cemiplimab.

70. The method of any one of claims 1-57, wherein the subject is undergoing or has undergone treatment for non-small cell lung cancer (NSCLC); and the treatment comprises administering to the subject one or more of pembrolizumab, nivolumab, atezolizumab, or durvalumab; or wherein the subject is undergoing or has undergone treatment for small cell lung cancer (SCLC); and the treatment comprises administering to the subject nivolumab.

71. The method of any one of claims 1-57, wherein the subject is undergoing or has undergone treatment for hepatocellular carcinoma; and the treatment comprises administering to the subject one or more of pembrolizumab or nivolumab.

72. The method of any one of claims 1-57, wherein the subject is undergoing or has undergone treatment for MSI-H or dMMR cancers; and the treatment comprises administering to the subject pembrolizumab.

73. The method of any one of claims 1-57, wherein the subject is undergoing or has undergone treatment for colorectal cancer (CRC); and the treatment comprises administering to the subject one or more of ipilimumab or nivolumab.

74. The method of any one of claims 1-57, wherein the subject is undergoing or has undergone treatment for metastatic urothelial carcinoma (mUC); and the treatment comprises administering to the subject one or more of nivolumab, pembrolizumab, atezolizumab, avelumab, or durvalumab.

75. The method of any one of claims 1-57, wherein the subject is undergoing or has undergone treatment for cervical cancer; and the treatment comprises administering to the subject pembrolizumab.

76. The method of any one of claims 1-57, wherein the subject is undergoing or has undergone treatment for SCCHN; and the treatment comprises administering to the subject one or more of pembrolizumab or nivolumab.

77. The method of any one of claims 1-57, wherein the subject is undergoing or has undergone treatment for gastric or gastroesophageal junction (GEJ) adenocarcinoma; and the treatment comprises administering to the subject pembrolizumab.

78. The method of any one of claims 1-57, wherein the subject is undergoing or has undergone treatment for renal cell carcinoma (RCC); and the treatment comprises administering to the subject one or more of ipilimumab or nivolumab.

79. The method of any one of claims 1-57, wherein the subject is undergoing or has undergone treatment for classical Hodgkin Lymphoma (CHL); and the treatment comprises administering to the subject one or more of pembrolizumab or nivolumab.

80. The method of any one of claims 1-57, wherein the subject is undergoing or has undergone treatment for primary mediastinal large B-cell lymphoma (PMBCL); and the treatment comprises administering to the subject pembrolizumab.

81. The method of any one of claims 1-57, wherein the subject is undergoing or has undergone treatment for triple-negative breast cancer (TNBC); and the treatment comprises administering to the subject atezolizumab.

82. The method of any one of claims 1-14, wherein the chemical entity or a pharmaceutically acceptable salt thereof, is administered by intravenous (IV) administration.
